(19) United States
(12) Patent Application Publication
(10) Pub. No.: US 2005/0059704 A1
(43) Pub. Date: Mar. 17, 2005

(54) Compositions useful for treating gastrointestinal motility disorders

(75) Inventors: Steven B. Landau, Wellesley, MA (US); Theodore T. Ashburn, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dynogen Pharmaceuticals, Inc., Waltham, MA (US)

(21) Appl. No.: 10/928,624

(22) Filed: Aug. 27, 2004

Related U.S. Application Data
(60) Provisional application No. 60/598,235, filed on Aug. 3, 2004.
(60) Provisional application No. 60/499,200, filed on Aug. 29, 2003.

(51) Int. Cl. A61K 31/4439

(52) U.S. Cl. 514/338

(57) ABSTRACT

The present invention relates to method of treating a gastrointestinal motility disorder in a subject in need of treatment comprising coadministering to said subject a first amount of a compound having 5-HT₃ receptor agonist activity or a pharmaceutically acceptable salt, hydrate or solvate thereof; and a second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) wherein the first and second amounts together comprise a therapeutically effective amount. In particular, the method is for treating GERD, including nocturnal GERD. The invention further relates to a method of treating nocturnal GERD comprising administering to a subject in need thereof a therapeutically effective amount of a compound having 5-HT₃ receptor agonist activity or a pharmaceutically acceptable salt, hydrate or solvate thereof. The invention further relates to a method of increasing esophageal motility in a subject in need thereof. The method of increasing esophageal motility can be achieved by administration of a compound having 5-HT₃ receptor agonist activity or a pharmaceutically acceptable salt, hydrate or solvate thereof. The coadministration can also be used to increase esophageal motility.

### RELATED APPLICATION

This application claims the benefit of U.S. Provisional Application No. 60/598,235, filed on Aug. 3, 2004, and of U.S. Provisional Application No. 60/499,200 filed on Aug. 29, 2003. The entire teachings of the above applications are incorporated herein by reference.

### BACKGROUND OF THE INVENTION

Gastrointestinal (GI) motility regulates the orderly movement of ingested material through the gut to ensure adequate absorption of nutrients, electrolytes and fluids. Appropriate transit through the esophagus, stomach, small intestine and colon depends on regional control of intraluminal pressure and several sphincters that regulate forward movement and prevent back-flow of GI contents. The normal GI motility pattern can be impaired by a variety of circumstances including disease and surgery.

Disorders of gastrointestinal motility can include, for example, gastroparesis and gastroesophageal reflux disease (GERD). Gastroparesis is the delayed emptying of stomach contents. Symptoms of gastroparesis include stomach upset, heartburn, nausea and vomiting. Acute gastroparesis can be caused by, for example, drugs, viral enteritis and hyperglycemia and is typically managed by treating the underlying disease rather than the motility disorder. The most common underlying disease resulting in gastroparesis is diabetes.

Gastroesophageal reflux is a physical condition in which stomach contents (e.g., stomach acid) reflux or flow back from the stomach into the esophagus. Frequent reflux episodes (e.g., two or more times per week) can result in a more severe problem known as GERD. The most common symptom of GERD is a burning sensation or discomfort behind the breastbone or sternum and is referred to as dyspepsia or heartburn. Dyspepsia can also mimic the symptoms of myocardial infarction or severe angina pectoris. Other symptoms of GERD include dysphagia, odynophagia, hemorrhage, water brash and respiratory manifestations such as asthma, recurrent pneumonia, chronic coughing, intermittent wheezing due to acid aspiration and/or stimulation of the vagus nerve, earache, hoarseness, laryngitis and pharyngitis.

Reflux episodes which result in GERD, can occur both during the daytime (i.e., when the subject is in a waking state) and at nighttime (i.e., when the subject is in a non-waking state). GERD occurring at nighttime is commonly referred to as nocturnal GERD. Nocturnal GERD is distinct from daytime or diurnal GERD not only in the timing of the reflux episode, but in the severity of the damage which occurs as a result of the reflux. More specifically, nocturnal GERD, can be particularly damaging to the pharynx and larynx and a strong association between nocturnal GERD and asthma exists. The increased damage associated with nocturnal GERD is due to a decrease in natural mechanisms which normally help protect against reflux (e.g., saliva production and swallowing), which occur when the patient is sleeping. This decrease leaves the esophagus more vulnerable to damage and can increase microaspiration. In addition, while asleep the body is in the recumbent position, eliminating the effect of gravity, which can clear gastric content from the esophagus. Sleep disorders are also associated with nocturnal GERD resulting in daytime sleepiness and a significant decrease in the overall quality of life.

On a chronic basis, GERD subjects the esophagus to ulcer formation or esophagitis and can result in more severe complications such as, esophageal erosion, esophageal obstruction, significant blood loss and perforation of the esophagus. Severe esophageal ulcerations occur in 20-30% of patients over age 65. In addition to esophageal erosion and ulceration, prolonged exposure of the esophageal mucosa to stomach acid can lead to a condition known as Barrett's Esophagus. Barrett's Esophagus is an esophageal disorder that is characterized by replacement of normal squamous epithelium with abnormal columnar epithelium. This change in tissue structure is clinically important not only as an indication of severe reflux, but as an indication of cancer.

Many factors are believed to contribute to the onset of GERD. A number of factors involve failure of the lower esophageal sphincter (LES) mechanism to work properly. These factors include, for example, increased transient lower esophageal sphincter relaxations (TLESR) and decreased lower esophageal sphincter (LES) resting tone. The LES is a physiologic, non-anatomic area involving the lower 3 centimeters of the esophagus and, like other smooth muscle sphincters in the body (e.g., anal and urinary), the LES is tonically contracted to prevent reflux. In a healthy person the muscle relaxes only during swallowing to allow food to pass and also on average three to four times and hour in a phenomenon known as TLESR. In GERD sufferers, the frequency of TLSER can be much higher, for example, as high as eight or more times an hour and weakness of the LES allows reflux to occur. Other factors which can contribute to GERD include delayed stomach emptying and ineffective esophageal clearance.

Therefore, the extent and severity of GERD depends not only on the presence of gastroesophageal reflux but on factors including the volume of gastric juice available to reflux, the potency of the refluxed material, the interval that the refluxed material remains in the esophagus and the ability of the esophageal tissue to withstand injury and to repair itself after injury.

Current methods to treat GERD include lifestyle changes such as weight loss, avoidance of certain foods that exacerbate the symptoms of GERD and avoidance of excessive bending. Elevation of the head of the bed helps reduce nocturnal reflux. While these avoidance strategies can be useful, the efficacy of lifestyle modification alone for the treatment of GERD is not supported.

Medications for the treatment of GERD include conventional antacids, for example, TUMS® and ROLAIDS® which provide only short term relief. H₂ receptor antagonists, for example, nizatidine (AXID®), ranitidine (ZANTAC®), famotidine (PEPCID® and PEPCID COMPLETE®), roxatidine (ROTANE® or ZORPEX®) and cimetidine (TAGAMET®), are more effective in controlling GERD symptoms, but do not treat the underlying disease. However, patients receiving H₂ receptor antagonists develop tolerance to the drugs rendering the drugs ineffective in their ability to inhibit acid secretion (Fackler et al., Gastroenterology, 122(3):625-632 (2002)).

More powerful secretory inhibitors, such as the proton pump inhibitors, for example, esomeprazole (NEXIM®), omeprazole (PRILOSEC® and RAPINEX®), lansoprazole (PREVACID®), rabeprazole (PARIET®, ACIPHEX®) and pantoprazole (PROTONIX®) are more effective than the H₂ receptor antagonists but are very expensive and their efficacy relies on inhibition of active proton pumps as stimulated by meals, thereby having little or no effect on the occurrence of nocturnal GERD.

Prokinetic drugs are another type of drug used in the treatment of gastrointestional motility disorders. Prokinetic drugs act to stimulate gastrointestinal motility. Stimulation can occur by direct action on smooth muscle or by an action on the myenteric plexus. The motor functions of the gastrointestinal tract are expressions of a balance at the level of smooth muscle cells between inhibitory mechanisms mainly regulated by dopamine and stimulatory events mainly regulated through the release of acetylcholine. Therefore gastrointestinal motility can be stimulated by dopamine antagonists such as metoclopramide and domperidone, or by substances which release acetylcholine such as metoclopramide or the 5-HT₄ receptor agonist, cisapride (PROPULSID®), or directly by cholinergic drugs which bind on muscarinic receptors of the smooth muscle cell such as bethanechol. Prokinetic drugs can both stimulate motility and coordinate the activity between different segments of the gastrointestinal tract. However, there are currently no prokinetic drugs available which are both effective and safe. For example, serious cardiac arrhythmias including ventricular tachycardia, ventricular fibrillation, torsades de pointes, and QT prolongation have been reported in patients taking the prokinetic of choice, cisapride. As a result, strict limitations have been imposed on the prescribing of this drug. Further, the use of the dopamine antagonists, metoclopramide and domperidone, is associated with lack of patient tolerability, undesirable CNS effects, such as diskinesia and undesirable cardiovascular effects, such as QT prolongation.

In view of the above, it is clear that none of the current agents address the multifactorial etiology of gastrointestinal motility disorders, such as GERD. Thus, a need exists for a new method of treating gastrointestinal motility disorders, such as GERD, which can effectively address the multifactorial etiology of the disorders.

### SUMMARY OF THE INVENTION

The invention relates to a method of treating a gastrointestinal motility disorder in a subject in need of treatment comprising coadministering to said subject a first amount of a compound having 5-HT₃ receptor agonist activity or a pharmaceutically acceptable salt, hydrate or solvate thereof and a second amount of at least one gastric acid suppressing agent, wherein the first and second amounts together comprise a therapeutically effective amount. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, hydrate or solvate thereof.

In one embodiment, the gastrointestinal motility disorder is GERD. In a particular embodiment, the GERD is nocturnal GERD.

In another embodiment, the gastrointestinal motility disorder is gastroparesis.

The invention further relates to a method of increasing esophageal motility in a subject in need thereof comprising coadministering to said subject a first amount of a compound having 5-HT₃ receptor agonist activity or a pharmaceutically acceptable salt, hydrate or solvate thereof and a second amount of at least one gastric acid suppressing agent, wherein the first and second amounts together comprise a therapeutically effective amount. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, hydrate or solvate thereof.

In one embodiment, the pharmaceutical composition for use in a method of increasing esophageal motility is used to treat a gastrointestinal motility disorder. In a specific embodiment, the gastrointestinal motility disorder is GERD. In a particular embodiment, the GERD is nocturnal GERD.

In certain embodiments, coadministration of a first amount of a compound having 5-HT₃ receptor agonist activity or a pharmaceutically acceptable salt, hydrate or solvate thereof and a second amount of at least one gastric acid suppressing agent such as an H₂ receptor antagonist or a pharmaceutically acceptable salt, hydrate or solvate thereof can result in an enhanced or synergistic therapeutic effect. For example, the combined effect of the first and second amounts can be greater than the additive effect resulting from separate administration of the first amount of the compound having 5-HT₃ receptor agonist activity or a pharmaceutically acceptable salt, hydrate or solvate thereof or the second amount of the gastric acid suppressing agent such as an H₂ receptor antagonist or a pharmaceutically acceptable salt, hydrate or solvate thereof.

The invention further relates to pharmaceutical compositions for use in therapy or prophylaxis, for example, in the treatment of a gastrointestinal motility disorder in a subject in need of treatment or for increasing esophageal motility in a subject in need thereof. The pharmaceutical composition comprises a first amount of a compound having 5-HT₃ receptor agonist activity or a pharmaceutically acceptable salt, hydrate or solvate thereof and a second amount of at least one gastric acid suppressing agent. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, hydrate or solvate thereof. The pharmaceutical compositions of the present invention can optionally contain a pharmaceutically acceptable carrier. The first amount of a compound having 5-HT₃ receptor agonist activity or a pharmaceutically acceptable salt, hydrate or solvate thereof and the second amount of at least one gastric acid suppressing agent (e.g., a proton pump inhibitor, an H₂ receptor antagonist or a pharmaceutically acceptable salt, hydrate or solvate thereof; or an acid pump antagonist or a pharmaceutically acceptable salt, hydrate or solvate thereof), can together comprise a therapeutically effective amount.

In one embodiment, the gastrointestinal motility disorder treated with a pharmaceutical composition is GERD. In a particular embodiment, the GERD is nocturnal GERD.

In another embodiment, the gastrointestinal motility disorder is gastroparesis.

In one embodiment, the pharmaceutical composition for use in a method of increasing esophageal motility is used to treat a gastrointestinal motility disorder. In a specific embodiment, the gastrointestinal motility disorder is GERD. In a particular embodiment, the GERD is nocturnal GERD.

The invention further relates to the use of a pharmaceutical composition comprising a first amount of a compound having 5-HT₃ receptor agonist activity or a pharmaceutically acceptable salt, hydrate or solvate thereof and a second amount of at least one gastric acid suppressing agent for the manufacture of a medicament for use in therapy or prophylaxis, for example, for the treatment of a gastrointestinal motility disorder in a subject in need of treatment or for increasing esophageal motility in a subject in need thereof. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, solvate or hydrate thereof. The pharmaceutical composition used for the manufacture of a medicament can optionally contain a pharmaceutically acceptable carrier. The first amount of a compound having 5-HT₃ receptor agonist activity or a pharmaceutically acceptable salt, hydrate or solvate thereof and the second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof), can together comprise a therapeutically effective amount.

The invention further relates to the use of a first amount of a compound having 5-HT₃ receptor agonist activity or a pharmaceutically acceptable salt, hydrate or solvate thereof and a second amount of at least one gastric acid suppressing agent for the manufacture of a medicament for use in therapy or prophylaxis, for example, for the treatment of a gastrointestinal motility disorder in a subject in need of treatment or for increasing esophageal motility in a subject in need thereof. In one embodiment, the gastric acid suppressing agent is selected from the group consisting of a proton pump inhibitor, an H₂ receptor antagonist and a pharmaceutically acceptable salt, hydrate or solvate thereof In another embodiment, the gastric acid suppressing agent is an acid pump antagonist or a pharmaceutically acceptable salt, solvate or hydrate thereof. The first amount of a compound having 5-HT₃ receptor agonist activity or a pharmaceutically acceptable salt, hydrate or solvate thereof and the second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof), can together comprise a therapeutically effective amount.

The invention also relates to a method of treating nocturnal GERD in a subject in need of treatment comprising administering to said subject a therapeutically effective amount of a compound having 5-HT₃ receptor agonist activity or a pharmaceutically acceptable salt, hydrate or solvate thereof.

The invention further relates to a pharmaceutical composition comprising a compound having 5-HT₃ receptor agonist activity or a pharmaceutically acceptable salt, hydrate or solvate thereof for use in the treatment of nocturnal GERD.

The invention further relates to the use of a compound having 5-HT₃ receptor agonist activity or a pharmaceutically acceptable salt, hydrate or solvate thereof for the manufacture of a medicament for the treatment of nocturnal GERD.

The invention also relates to a method of increasing esophageal motility in a subject in need of thereof comprising administering to said subject a therapeutically effective amount of a compound having 5-HT₃ receptor agonist activity or a pharmaceutically acceptable salt, hydrate or solvate thereof.

In one embodiment the method of increasing esophageal motility is used to treat a gastrointestinal motility disorder. In a specific embodiment, the gastrointestinal motility disorder is GERD. In a particular embodiment, the GERD is nocturnal GERD.

The invention further relates to a pharmaceutical composition comprising a compound having 5-HT₃ receptor agonist activity or a pharmaceutically acceptable salt, hydrate or solvate thereof a for use in increasing esophageal motility in a subject in need of thereof.

The invention further relates to the use of a compound having 5-HT₃ receptor agonist activity or a pharmaceutically acceptable salt, hydrate or solvate thereof for the manufacture of a medicament for increasing esophageal motility in a subject in need of thereof.

### BRIEF DESCRIPTION OF THE DRAWINGS

FIG. 1 is a bar graph showing the effects of intravenous administration of MKC-733 at the indicated dose in cats on Lower Esophageal Sphincter Pressure (LESP) before (naive) and after chronic omeprazole administration (n=5, 5, 3 for the vehicle, 1.0 mg/kg MKC-733 and 10 mg/kg MKC-733 treatments, respectively). Data from each animal were normalized to its MKC-733 vehicle response from the naive treatment period.

FIG. 2 is a bar graph showing the effects of intravenous administration of MC-733 at the indicated dose on Lower Esophageal Sphincter Pressure (LESP) before (naive) and after chronic omeprazole administration in cats (n=5, 5, 3 for the vehicle, 1.0 mg/kg MKC-733 and 10 mg/kg MKC-733 treatments, respectively). Data from each animal in the naive group were normalized to their respective MKC-733 vehicle response from the naive treatment period. Data from each animal in the omeprazole pre-treated group were normalized to their respective MKC-733 vehicle response for that treatment period.

FIG. 3 is a bar graph showing the effects of intravenous administration of MKC-733 at the indicated dose in cats on the percentage of time during a gastroesophageal reflux event that lower esophageal pH is greater than 4.0 (n=5, 5, 2 for the vehicle, 1.0 mg/kg MKC-733 and 10 mg/kg MKC-733 treatments, respectively).

FIG. 4 is a bar graph showing the effects of intravenous administration of MKC-733 at the indicated dose in cats on the nadir values of lower esophageal pH that occur during a gastroesophageal reflux event (n=5, 5, 2 for the vehicle, 1.0 mg/kg MKC-733 and 10 mg/kg MKC-733 treatments, respectively).

FIG. 5 is a bar graph showing the effects of intravenous administration of MKC-733 at the indicated dose in cats on the esophageal peristaltic peak contraction pressure (n=5, 5, 3 for the vehicle, 1.0 mg/kg MKC-733 and 10 mg/kg MKC-733 treatments, respectively).

### DETAILED DESCRIPTION OF THE INVENTION

The invention relates to a method of treating a gastrointestinal motility disorder in a subject in need of treatment. In one embodiment, the gastrointestinal motility disorder is GERD. In a particular embodiment, the GERD is nocturnal GERD. In another embodiment, the gastrointestinal motility disorder is gastroparesis.

The invention also relates to a method of increasing esophageal motility in a subject in need of thereof comprising administering to said subject a therapeutically effective amount of a compound having 5-HT₃ receptor agonist activity or a pharmaceutically acceptable salt, hydrate or solvate thereof.

In one embodiment the method of increasing esophageal motility is used to treat a gastrointestinal motility disorder. In a specific embodiment, the gastrointestinal motility disorder is GERD. In a particular embodiment, the GERD is nocturnal GERD.

Serotonin and 5-HT₃ Receptor Agonists

The neurotransmitter serotonin was first discovered in 1948 and has subsequently been the subject of substantial scientific research. Serotonin, also referred to as 5-hydroxytryptamine (5-HT), acts both centrally and peripherally on discrete 5-HT receptors. Currently, fourteen subtypes of serotonin receptors are recognized and delineated into seven families, 5-HT₁ through 5-HT₇. These subtypes share sequence homology and display some similarities in their specificity for particular ligands. While these receptors all bind serotonin, they initiate different signalling pathways to perform different functions. For example, serotonin is known to activate submucosal intrinsic nerves via 5-HT_{1P} and 5-HT₄ receptors, resulting in, for example, the initiation of peristaltic and secretory reflexes. However, serotonin is also known to activate extrinsic nerves via 5-HT₃ receptors, resulting in, for example, the initiation of bowel sensations, nausea, bloating and pain. A review of the nomenclature and classification of the 5-HT receptors can be found in Neuropharm., 33: 261-273 (1994) and Pharm. Rev., 46:157-203 (1994).

5-HT₃ receptors are ligand-gated ion channels that are extensively distributed on enteric neurons in the human gastrointestinal tract, as well as other peripheral and central locations. Activation of these channels and the resulting neuronal depolarization have been found to affect the regulation of visceral pain and colonic transit. Antagonism of the 5-HT₃ receptors has the potential to influence sensory and motor function in the gut.

As used herein, 5-HT₃ receptor refers to naturally occurring 5-HT₃ receptors (e.g., mammalian 5-HT₃ receptors (e.g., human (Homo sapiens) 5-HT₃ receptors, murine (e.g., rat, mouse) 5-HT₃ receptors)) and to proteins having an amino acid sequence which is the same as that of a corresponding naturally occurring 5-HT₃ receptor (e.g., recombinant proteins). The term includes naturally occurring variants, such as polymorphic or allelic variants and splice variants.

As used herein, the term a compound having 5-HT₃ receptor agonist activity refers to a substance (e.g., a molecule, a compound) which promotes (induces or enhances) at least one function characteristic of a 5-HT₃ receptor. In one embodiment, the compound having 5-HT₃ receptor agonist activity binds the 5-HT₃ receptor (i.e., is a 5-HT₃ receptor agonist). In certain embodiments, the agonist is a partial agonist. Partial agonist, as used herein, refers to an agonist which no matter how high of a concentration is used, is unable to produce maximal activation of the 5-HT₃ receptor. A compound having 5-HT₃ receptor agonist activity (e.g., a 5-HT₃ receptor agonist) can be identified and activity assessed by any suitable method. For example, the binding affinity of a 5-HT₃ receptor agonist to the 5-HT₃ receptor can be determined by the ability of the compounds to displace [³H]granisetron from rat cortical membranes (Cappelli et al., J. Med. Chem., 42(9): 1556-1575 (1999)). In addition, the agonist activity of the compounds can be assessed in vitro on, for example, the 5-HT₃ receptor-dependent [¹⁴C]guanidinium uptake in NG 108-15 cells as described in Cappelli et al.

In a particular embodiment, the compounds having 5-HT₃ receptor agonist activity are thieno[3,2-b]pyridine derivatives such as those described in U.S. Pat. No. 5,352,685, the entire content of which is incorporated herein by reference.

In a specific embodiment, the compounds having 5-HT₃ receptor agonist activity are represented by Formula I: 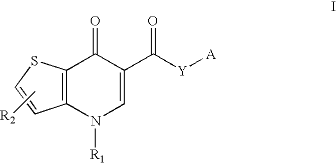
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 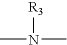
   wherein R₃ represents hydrogen or a C₁-C₆alkyl group; and
A is represented by 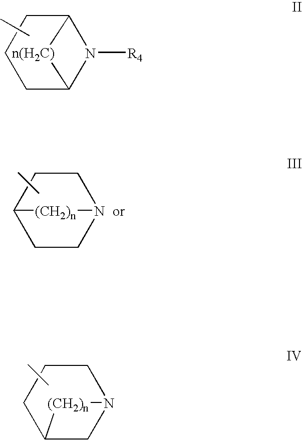
   wherein:
n is an integer from 1 to about 4;
R₄ represents hydrogen, a C₁-C₆ alkyl group, a C₃-C₈ cycloalkyl group or a C₇-C₁₈ aralkyl group;
   or a pharmaceutically acceptable salt, solvate, hydrate or N-oxide derivative thereof.

It is understood that when R₁ of Formula I is hydrogen, compounds having the tautomeric form represented by Formula IA are included within the definition of Formula I.

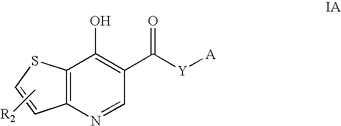

Likewise, it is understood that Formula IA includes the tautomeric form represented by Formula I when R₁ is hydrogen.

In one embodiment, the compounds represented by Formula I can be N-oxide derivatives.

In another embodiment of Formula I, Y represents —O— or 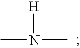
R₁ represents hydrogen, a C₁-C₆ alkyl group, a C₆-C₁₂ aryl group, or a C₇-C₁₈ aralkyl group;
R₂ represents hydrogen, a C₁-C₆ alkyl group or halogen; and
A is represented by 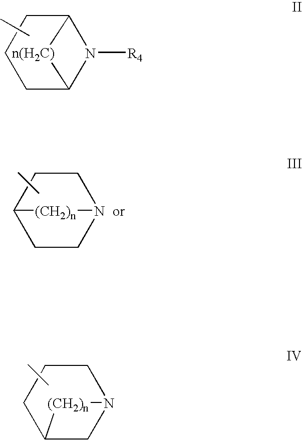
   wherein:
n is 2 or 3; and
R₄represents a C₁-C₆ alkyl group.

In a particular embodiment, the compounds having 5-HT₃ receptor agonist activity are represented by Formula I, wherein R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

In a particularly preferred embodiment, the compound having 5-HT₃ receptor agonist activity is represented by structural Formula V: 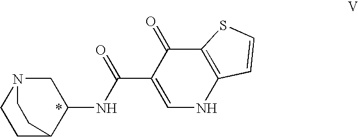
or a pharmaceutically acceptable salt, solvate or hydrate thereof.

In a particular embodiment, the compound represented by Formula I is an N-oxide derivative.

In a particularly preferred embodiment, the compound of Formula V has the (R) configuration at the chiral carbon atom which is designated with an asterisk (*). The chemical name of the compound set forth in Formula V having the (R) configuration at the designated chiral carbon is: (R)—N-1-azabicyclo[2.2.2]oct-3-yl-4,7-dihydro-7-oxothieno[3,2-b]pyridine-6-carboxamide. When the compound is in the form of the monohydrochloride, it is known as MKC 733 (CAS Number: 194093-42-0). When the compound of Formula V has the (S) configuration at the chiral carbon atom designated with an asterisk (*), the chemical name is (S)—N-1-azabicyclo[2.2.2]oct-3-yl-4,7-dihydro-7-oxothieno[3,2-b]pyridine-6-carboxamide.

It is understood that structural Formula V includes the tautomeric form depicted by Formula VA:

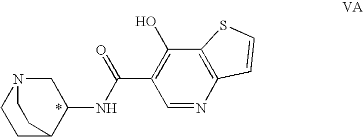

Likewise, it is understood that Formula VA includes the tautomeric form represented by Formula V.

For example, when Formula V has the (R) configuration at the designated chiral carbon the compound is referred to as: (R)—N-1-azabicyclo[2.2.2]oct-3-yl-4,7-dihydro-7-oxothieno[3,2-b]pyridine-6-carboxamide which is understood to include the tautomeric form: (R)—N-1-azabicyclo[2.2.2]oct-3-yl)-7-hydroxythieno[3,2-b]pyridine-6-carboxamide.

Likewise, when Formula VA has the (R) configuration at the designated chiral carbon the compound is referred to as: (R)—N-1-azabicyclo[2.2.2]oct-3-yl)-7-hydroxythieno[3,2-b]pyridine-6-carboxamide, which is understood to include the tautomeric form: (R)—N-1-azabicyclo[2.2.2]oct-3-yl-4,7-dihydro-7-oxothieno[3,2-b]pyridine-6-carboxamide.

In another embodiment, the compounds having 5-HT₃ receptor agonist activity are condensed thiazole derivatives such as those described in U.S. Pat. No. 5,565,479, the entire content of which is incorporated herein by reference.

In a particular embodiment, the compounds having 5-HT₃ receptor agonist activity are represented by Formula VI or a pharmaceutically acceptable salt, solvate or hydrate thereof: 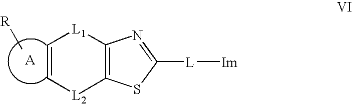
wherein:
R represents hydrogen, halogen, hydroxyl, a C₁-C₆ alkoxy group, carboxy, a C₁-C₆ alkoxycarbonyl group, nitro, amino, cyano or protected hydroxyl; 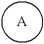
   is a phenyl ring or a naphthalene ring;
L is a direct bond or a C₁-C₆ alkylene group;
L₁ and L₂ are defined so that one is a direct bond and the other is:
   a) a C₁-C₆ alkylene group optionally containing an interrupting oxygen or sulfur atom therein;
   b) an oxygen atom or sulfur atom; or
   c) a C₁-C₆ alkenylene group;
Im represents a group having the formula: 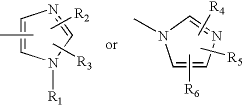
   wherein:
R₁-R₆ are the same or different each representing hydrogen or a C₁-C₆ alkyl group.

In a further embodiment, the compound according to Formula VI, 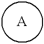
is a phenyl ring, L₁ is a direct bond and L₂ is an alkylene group or alkenylene group.

In a particularly preferred embodiment, the compound having 5-HT₃ receptor agonist activity is represented by structural Formula VII: 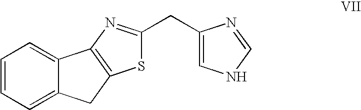
or a pharmaceutically acceptable salt, solvate, or hydrate thereof. This compound is commonly referred to in the art as YM 31636. The chemical name of the compound set forth in Formula VII is: 2-(1H-imidazol-4-ylmethyl)-8H-indeno[1,2-d]thiazole.
Gastric Acid Suppressing Agents

Gastric acid suppressing agents are agents that suppress gastric acid secretion in the gastrointestinal tract. Agents that act as inhibitors (e.g., antagonists) of any one of the histamine, gastrin or muscarinic receptors present on the surface of parietal cells can suppress gastric acid secretion. Other agents which suppress gastric acid secretion work by inhibiting the enzyme H+−K+ATPase, commonly referred to as the proton pump, found in parietal cells.

Antagonists of the histamine receptor are commonly referred to as H₂ receptor antagonists and include agents such as cimetidine and ranitidine. Antagonists of the muscarinic receptor include agents such as pipenzepine and propantheline. Antagonists of the gastrin receptor include agents such as proglumide. Inhibitors of H+−K+ATPase enzyme system include both reversible and irreversible inhibitors such as esomeprazole (NEXIUM®) and soraprazan or AZD0865, respectively.

Inhibitors of H+−K+ATPase (Proton Pump)

Inhibitors of H+−K+ATPase are compounds which can be used to treat gastrointestinal diseases by inhibiting the gastric enzyme H+−K+ATPase and thereby regulating acidity in gastric juices. More specifically, these inhibitors suppress gastric acid secretion, the final step of acid production, by specific inhibition of H+−K+ATPase present in gastric parietal cells. Inhibitors of H+−K+ATPase (proton pump) can bind irreversibly and/or reversibly. Agents referred to as Proton Pump Inhibitors (PPIs) typically include irreversible inhibitors. Agents referred to as Acid Pump Antagonists (APAs) typically include reversible inhibitors.

Proton Pump Inhibitors (PPIs) include benzimidazole compounds, for example, esomeprazole (NEXIUM®), omeprazole (PRILOSEC® and RAPINEX® (oral suspension of omeprazole in combination with an antacid)), lansoprazole (PREVACID®), rabeprazole (PARIET®, ACIPHEX®) and pantoprazole (PROTONIX®). These proton pump inhibitors contain a sulfinyl group situated between substituted benzimidazole and pyridine rings. At neutral pH, esomeprazole, omeprazole, lansoprazole, rabeprazole and pantoprazole are chemically stable, lipid soluble, weak bases that are devoid of inhibitory activity. These uncharged weak bases reach parietal cells from the blood and diffuse into the secretory canaliculi, where the drugs become protonated and thereby trapped. The protonated species rearranges to form a sulfonic acid and a sulfonamide, the latter species capable of interacting with sulfhydryl groups of H+−K+ATPase. Full inhibition occurs with two molecules of inhibitor per molecule of enzyme. The specificity of the effects of proton pump inhibitors is believed to derive from: a) the selective distribution of H+−K+ATPase; b) the requirement for acidic conditions to catalyze generation of the reactive inhibitor; and c) the trapping of the protonated drug and the cationic sulfonamide within the acidic canuliculi and adjacent to the target enzyme. Goodman & Gilman's The Pharmacological Basis of Therapeutics, 9^{th} Edition, pp. 901-915 (1996).

However, due to the requirement for accumulation in the acid space of the parietal cell, acid secretion is necessary for the efficacy of the PPI type drugs. The plasma half life of PPI type drugs has been found to be between 60 to 90 minutes. All acid pumps are not active at any one time, rather only about 75% are active on the average during the time the drug is present in the blood following oral administration. As this is the case, it has been reported that employing a currently used once-a-day oral administration therapy, the maximal inhibition of stimulated acid output was approximately 66%. This is due to a combination of the short plasma half life of the drug, the limited number of acid pumps active during presentation of the drug, and the turn-over of acid pumps. Therefore, in present practice it is not possible to control nighttime acid secretion using any PPI regimen since the agents can only inhibit active proton pumps, resulting in a patient population with nocturnal acid breakthrough and nocturnal GERD. The pharmaceutical compositions and methods of coadministration of the present invention can address this need.

The Acid Pump Antagonists (APAs) differ from the PPIs in the way in which they inhibit H+−K+ATPase. For example, acid induced transformation is not necessary and enzyme kinetics typically show reversible binding to the enzyme for APAs. In addition, APAs can work faster than the PPIs following administration. Suitable APAs include, but are not limited to those described in U.S. Pat. No. 6,132,768 to Sachs et al. and U.S. Published application No. US2004/0058896 A1 the contents of each of which are incorporated herein by reference. Examples of suitable APAs include, but are not limited to, YH1885 (Yuhan Co.); CS-526 (Sankyo); AZD0865 (AstraZeneca); Soraprazan (Altana AG):((7R,8R,9R)-2,3-dimethyl-8-hydroxy-7-(2-methoxyethoxy)-9-phenyl-7,8,9,10-tetrahydroimidazo[1,2-h]-[1,7]naphthyridine)); (7R,8R,9R)-2,3-dimethyl-7,8-dihydroxy-9-phenyl-7,8,9,10-tetrahydroimidazo[1,2-h][1,7]naph-thyridine; 7,8-dihydroxy-2,3-dimethyl-9-phenyl-7,8,9,10-tetrahydroimidazo[1,2-h][1,7]naphthyridine; 7-hydroxy-2,3-dimethyl-9-phenyl-7,8,9,10-tetrahydroimidazo[1,2-h][1,7]naphthyridine; 9-(2-chlorophenyl)-7-hydroxy-2,3-dimethyl-7,8,9,10-tetrahydroimidazo[1,2-h][1,7]naphthyridine; 9-(2,6-dichlorophenyl)-7-hydroxy-2,3-dimethyl-7,8,9,10-tetrahydroimidazo[1,2-h] [1,7]naphthyridine; 9-(2-trifluoromethylphenyl)-7-hydroxy-2,3-dimethyl-7,8,9,10-tetrahydroimidazo[1,2-h][1,7]naphthyridine; 8-hydroxy-2,3-dimethyl-9-phenyl-7,8,9, 1 0-tetrahydroimidazo[1,2-h][1,7]naphthyridin-7-one; (8R,9R)-3-formyl-8-hydroxy-2-methyl-7-oxo-9-phenyl-7,8,9,10-tetrahydroimidazo[1,2-h][1,7]naphthyridine; (7R,8R,9R)-3-hydroxymethyl-7,8-dihydroxy-2-methyl-9-phenyl-7,8,9,10-tetrahydroimidazo[1,2-h][1,7]-naphthyridine; (7S,8R,9R)-7,8-isopropylidenedioxy-2,3-dimethyl-9-phenyl-7,8,9,10-tetrahydro-imidazo[1,2-h][1,7]naphthyridine; 8,9-trans-8-hydroxy-3-hydroxymethyl-2-methyl-9-phenyl-7H-8,9-dihydropyrano[2,3-c]imidazo[1,2-a]-pyridine; 8,9-cis-8-hydroxy-3-hydroxymethyl-2-methyl-9-phenyl-7H-8,9-dihydropyrano[2,3-c]imidazo [1,2-a]-pyridine; 8,9-trans-3-hydroxymethyl-2-methoxy-2-methyl-9-phenyl-7H-8,9-dihydro-pyrano-[2,3-c]imidazo[1,2-a]-pyridine; 8,9-cis-3-hydroxymethylmethoxy-2-methyl-9-phenyl-7H-8,9-dihydropyrano[2,3-c]imidazo[1,2-a]-pyridine; 8,9-trans-8-ethoxy-3-hydroxymethyl-2-methyl-9-phenyl-7H-8,9-dihydropyrano[2,3-c]imidazo[1,2-a]-pyridine; 8-hydroxy-7-oxo-9-phenyl-2,3-dimethyl-7H-8,9-dihydropyrano[2,3-c]imidazo[1,2-a]pyridine; 7,8-dihydroxy-9-phenyl-2,3-dimethyl-7H-8,9-dihydropyrano[2,3-c]imidazo[1,2-a]pyridine; 7-hydroxy-9-phenyl-2,3-dimethyl-7H-8,9-dihydropyrano[2,3-c]imidazo[1,2-a]pyridine; (7R,8R,9R)-2,3-dimethyl-8-hydroxy-7-methoxy-9-phenyl-7,8,9,10-tetrahydroimidazo[1,2-h][1,7]naphthyridine; (7S,8S,9S)-2,3-dimethyl-8-hydroxy-7-methoxy-9-phenyl-7,8,9,10-tetrahydroimidazo[1,2-h][1,7]naphthyridine; (7S,8R,9R)-2,3-dimethyl-8-hydroxy-7-methoxy-9-phenyl-7,8,9,10-tetra-hydroimidazo [1,2-h][1,7]naphthyridine; (7R,8S,9S)-2,3-dimethyl-8-hydroxy-7-methoxy-9-phenyl-7,8,9,10-tetrahydroimidazo[1,2-h][1,7]naphthyridine; (7R,8R,9R)-2,3-dimethyl-7-ethoxy-8-hydroxy-9-phenyl-7,8,9,10-tetrahydroimidazo[1,2-h]-[1,7]naphthyridine; (7S,8R,9R)-2,3-dimethyl-7-ethoxy-8-hydroxy-9-phenyl-7,8,9,10-tetrahydroimidazo[1,2-h][1,7]naphthyridine; (7S ,8S,9S)-2,3-dimethyl-8-hydroxy-7-(2-methoxyethoxy)-9-phenyl-7,8,9,10-tetrahydroimidazo[1,2-h][1,7]naphthyridine; (7S,8R,9R)-2,3-dimethyl-8-hydroxy-7-(2-methoxyethoxy)-9-phenyl-7,8,9,10-tetrahydroimidazo[1,2-h][1,7]naphthyridine; (7R,8S,9S)-2,3-dimethyl-8-hydroxy-7-(2-methoxyethoxy)-9-phenyl-7,8,9,10-tetrahydroimidazo[1,2-h]-[1,7]naphthyridine; (7S,8R,9R)-2,3-dimethyl-8-hydroxy-9-phenyl-7-(2-propoxy)-7,8,9,10-tetrahydro-imidazo[1,2-h][1,7]naphthyridine; (7R,8R,9R)-2,3-dimethyl-7,8-dimethoxy-9-phenyl-7,8,9,10-tetrahydroimidazo[1,2-h][1,7]naphthyridine; (7R,8R,9R)-2,3-dimethyl-8-hydroxy-7-(2-methylthioethyloxy)-9-phenyl-7,8,9,10-tetrahydroimidazo[1,2-h][1,7]naphthyridine; (7S ,8R,9R)-2,3-dimethyl-8-hydroxy-7-(2-methylthioethyloxy)-9-phenyl-7,8,9,10-tetrahydroimidazo[1,2-h][1,7]naphthyridine; (7R,8R,9R)-2,3-dimethyl-8-hydroxy-7-(2-methylsulfinylethoxy)-9-phen-yl-7,8,9,10-tetrahydroimidazo-[1,2-h][1,7]naphthyridine; (7S,8R,9R)-2,3-dimethyl-8-hydroxy-7-(2-methylsulfinylethoxy)9-phenyl-7,8,9,10-tetrahydroimidazo-[1,2-h][1,7]naphthyridine; (7R,8R,9R)-2,3-dimethyl-8-hydroxy-7-(ethylthio)-9-phenyl-7,8,9,10-tetrahydroimidazo[1,2-h][1,7]-naphthyridine; (7S,8R,9R)-2,3-dimethyl-8-hydroxy-7-(ethylthio)-9-phenyl-7,8,9,10-tetrahydroimidazo[1,2-h][1,7]-naphthyridine; (7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine; (7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine; AU-461: 2-[1-(2-methyl-4-methoxyphenyl)-6-(2,2,2-trifluoroethoxy)-2,3-dihydro-1H-pyrrolo-[3,2-c]quinolin4-ylamino]-1-ethanol; DBM-819: 3-[1-(4-methoxy-2-methylphenyl)-6-methyl-2,3-dihydro-1H-pyrrolo-[3,2-c]quinolin-4-ylamino] 1-propanol; KR-60436: 2-[1-(4-methoxy-2-methylphenyl)-6-(trifluoromethoxy)-2,3-dihydro-1H-pyrrolo[3,2-c]quinolin-4-ylamino]ethanol; R-105266; YJA-20379-8: (+)-1-[8-ethoxy-4-[(1 (R)-phenylethyl)amino]-1,7-naphthyridin-3-yl]-1-butanone; 8-(2-methoxycarbonylamino-6-methylbenzylamino)-2,3-dimethylimidazo-[1,2-a]pyridine; 3-hydroxymethyl-8-(2-methoxycarbonylamino-6-methylbenzylamino)-2-methylimidazo [1,2-a]-pyridine; 3-hydroxymethyl-8-(2-methoxycarbonylamino-6-methylbenzyloxy)-2-methylimidazo[1,2-a]-pyridine; 8-(2-methoxycarbonylamino-6-methylbenzyloxy)-2,3-dimethylimidazo[1,2-a]pyridine; 8-(2-tert-butoxycarbonylamino-6-methylbenzylamino)-2,3-dimethylimidazo[1,2-a]pyridine; 8-(2-tert-butoxycarbonylamino-6-methylbenzyloxy)-2,3-dimethylimidazo[1,2-a]pyridine; 8-(2-ethoxycarbonylamino-6-methylbenzylamino)-2,3-dimethylimidazo[1,2-a]pyridine; 8-(2-isobutoxycarbonylamino-6-methyl dimethylimidazo[1,2-a]pyridine; 8-(2-isopropoxycarbonylamino-6-methylbenzylamino)-2,3-dimethylimidazo[1,2-a]pyridine; 8-(2-tert-butoxycarbonylamino-6-methylbenzylamino)-3-hydroxymethyl-2-methylimidazo[1,2-a]-pyridine; 8-(2-tert-butoxycarbonylamino-6-methylbenzyloxy)-3-hydroxymethyl-2-methylimidazo[1,2-a]-pyridine; 8-(2-[(2-methoxyethoxy)carbonylamino]-6-methylbenzyloxy)-2-methylimidazo-[1,2-a]pyridine-3-methanol; 8(2-[(2-methoxyethoxy)carbonylamino]-6-methylbenzylamino)-2-methylimidazo [1,2-a]-pyridine-3-methanol; 8-(2-[(2-methoxyethoxy)carbonylamino]-6-methylbenzylamino]-2,3-dimethylimidazo[1,2-a]-pyridine; 8-(2-[(2-methoxyethoxy)carbonylamino]-6-methylbenzyloxy)-2-methylimidazo[1,2-a]pyridine-3-methanol; 8-(2-[(2-methoxyethoxy)carbonylamino]-6-methylbenzylbenzyloxy-2,3-dimethylimidazo[1,2-a]pyridine; 3-hydroxymethyl-2-methyl-8-benzyloxyimidazo[1,2-a]pyridine; 3-hydroxymethyl-2-trifluoromethyl-8-benzyloxyimidazo[1,2-a]pyridine; 1,2-dimethyl-3-cyanomethyl-8-benzyloxyimidazo[1,2-a]pyridine; 2-methyl-3-cyanomethyl-8-benzyloxyimidazo-[1,2-a]pyridine; 3-butyryl-8-methoxy-4-(2-methylphenylamino)quinoline; 3-butyryl-8-hydroxyethoxy-4-(2-methylphenylamino)quinoline; 3-hydroxymethyl-2-methyl-9-phenyl-7H-8,9-dihydropyrano[2,3-c]-imidazo[1,2-a]pyridine; 3-hydroxymethyl-2-methyl-9-(4-fluorophenyl)-7H-8,9-dihydropyrano[2,3-c]imidazo[1,2-a]pyridine; (+)-3-hydroxymethyl-2-methyl-9-phenyl-7H-8,9-dihydropyrano[2,3-c]imidazo[1,2-a]pyridine; (−)-3-hydroxymethyl-2-methyl-9-phenyl-7H-8,9-dihydropyrano[2,3-c]imidazo[1,2-a]pyridine; 8-(2-ethyl-6-methylbenzylamino)-3-(hydroxymethyl)-2-methylimidazo-[1,2-a]pyridine-6-carboxamide; N-(2-hydroxyethyl)-8-(2,6-dimethylbenzylamino)-2,3-dimethylimidazo[1,2-a]pyridine-6-carboxamide; 8-(2-ethyl-6-methylbenzylamino)-2,3-dimethylimidazo[1,2-a]pyridine-6-carboxamide; 8-(2-ethyl-6-methylbenzylamino)-N,2,3-trimethylimidazo[1,2-a]pyridine-6-carboxamide; 8-(2,6-dimethylbenzylamino)-2,3-dimethylimidazo[1,2-a]pyridine-6-carboxamide; 8-(2-ethyl-4-fluoro-6-methylbenzylamino)-2,3-dimethylimidazo[1,2-a]pyridine-6-carboxamide; 8-(4-fluoro-2,6-dimethylbenzylamino)-2,3-dimethylimidazo[1,2-a]pyridine-6-carboxamide; 8-(2,6-diethylbenzylamino)-2,3-dimethylimidazol[1,2-a]pyridine-4-carboxamide; 8-(2-ethyl-6-methylbenzylamino)-N-(2-hydroxyethyl)-2,3-dimethylimidazo [1,2-a]pyridine-6-carboxamide; 8-(2-ethyl-6-methylbenzylamino)-N-(2-methoxyethyl)-2,3-dimethylimidazo[1,2-a]pyridine-6-carboxamide; 8-(2-ethyl-6-methylbenzylamino)-3-(hydroxymethyl)-2-methylimidazo[1,2-a]pyridine-6-carboxamide; N-(2-hydroxyethyl)-8-(2,6-dimethylbenzylamino)-2,3-dimethylimidazo[1,2-a]pyridine-6-carboxamide; 8-(2-ethyl-6-methylbenzylamino)-2,3-dimethylimidazo[1,2-a]pyridine-6-carboxamide; 8-(2-ethyl-6-methylbenzylamino)-N,2,3-trimethylimidazo-[1,2-a]pyridine-6-carboxamide; 8-(2,6-dimethylbenzylamino)-2,3-dimethylimidazo-[1,2-a]pyridine-6-carboxamide; 8-(2-ethyl-4-fluoro-6-methylbenzylamino)-2,3-dimethylimidazo[1,2-a]pyridine-6-carboxamide; 8-(4-fluoro-2,6-dimethylbenzylamino)-2,3-dimethylimidazo [1,2-a]-pyridine-6-carboxamide; 8-(2,6-diethylbenzylamino)-2,3-dimethylimidazo-[1,2-a]pyridine-6-carboxamide; 8-(2-ethyl-6-methylbenzylamino)-N-(2-hydroxyethyl)-2,3-dimethylimidazo[1,2-a]pyridine-6-carboxamide and 8-(2-ethyl-6-methylbenzylamino)-N-(2-methoxyethyl)-2,3-dimethylimidazo-[1,2-a]pyridine-6-carboxamide.

H₂ Receptor Antagonists

H₂ receptor antagonists inhibit gastric acid secretion elicited by histamine, other H₂ receptor agonists, gastrin, and, to a lesser extent, muscarinic agonists. H₂ receptor antagonists also inhibit basal and nocturnal acid secretion.

H₂ receptor antagonists competitively inhibit the interaction of histamine with H₂ receptors. They are highly selective and have little or no effect on H₁ receptors. Although H₂ receptors are present in numerous tissues, including vascular and bronchial smooth muscle, they appear to have a minimal role in modulating physiological functions other than gastric acid secretion. H₂ receptor antagonists reduce both the volume of gastric juice secreted and its hydrogen ion concentration. However, despite their good antisecretory properties, H₂ receptor antagonists are not unanimously recognized as gastroprotective agents. H₂ receptor antagonists include nizatidine (AXID®), ranitidine (ZANTAC®), famotidine (PEPCID COMPLETE®, PEPCID®), roxatidine (ROTANE® or ZORPEX®) and cimetidine (TAGAMET®). Goodman & Gilman's The Pharmacological Basis of Therapeutics, 9^{th} Edition, pp. 901-915 (1996). However, patients receiving H₂ receptor antagonists develop tolerance to the drugs rendering the drugs ineffective in their ability to inhibit acid secretion (Fackler et al., Gastroenterology, 122(3):625-632 (2002)).

Gastrointestinal motility disorders, as used herein, refers to disorders of the gastrointestinal tract wherein the normal orderly movement of ingested material through the gastrointestinal tract is impaired. Gastrointestinal motility disorders include, for example, gastroparesis and gastroesophageal reflux disease (GERD).

Gastroparesis is the delayed emptying of stomach contents. Symptoms of gastroparesis include stomach upset, heartburn, nausea and vomiting. Acute gastroparesis can be caused by, for example, drugs, viral enteritis and hyperglycemia and is typically managed by treating the underlying disease rather than the motility disorder. The most common underlying disease causing gastroparesis is diabetes.

Gastroesophageal reflux is a physical condition in which stomach contents (e.g., stomach acid) reflux or flow back from the stomach into the esophagus. Frequent reflux episodes (e.g., two or more times per week) can result in a more severe problem known as GERD. The most common symptom of GERD is a burning sensation or discomfort behind the breastbone or sternum and is referred to as dyspepsia or heartburn. Dyspepsia can also mimic the symptoms of myocardial infarction or severe angina pectoris. Other symptoms of GERD include dysphagia, odynophagia, hemorrhage, water brash and respiratory manifestations such as asthma, recurrent pneumonia, chronic coughing, intermittent wheezing due to acid aspiration and/or stimulation of the vagus nerve, earache, hoarseness, laryngitis and pharyngitis.

Reflux episodes which result in GERD, can occur during the daytime (i.e., when the subject is in a waking state) and/or at nighttime (i.e., when the subject is in a non-waking state). GERD occurring at nighttime is commonly referred to as Nocturnal GERD. Nocturnal GERD is distinct from daytime or diurnal GERD not only in the timing of the reflux episode, but in the severity of the damage which occurs as a result of the reflux. Many patients experience both nocturnal and diurnal symptoms of GERD. As used herein the treatment of nocturnal GERD encompasses the treatment of patients having reflux episodes occurring at night, which may or may not be accompanied by daytime symptoms. More specifically, nocturnal GERD, can be particularly damaging to the pharynx and larynx and a strong association between nocturnal GERD and asthma exists. The increased damage associated with nocturnal GERD is due to a decrease in natural mechanisms which normally help protect against reflux (e.g., saliva production and swallowing), which occur when the patient is sleeping. This decrease leaves the esophagus more vulnerable to damage and can increase microaspiration. In addition, while asleep the body is in the recumbent position, eliminating the effect of gravity, which can clear gastric content from the esophagus. Sleep disorders are also associated with nocturnal GERD resulting in daytime sleepiness, as are chronic cough, chronic throat clearing and a significant decrease in the overall quality of life.

On a chronic basis, GERD subjects the esophagus to ulcer formation or esophagitis and can result in more severe complications such as, esophageal erosion, esophageal obstruction, significant blood loss and perforation of the esophagus. Severe esophageal ulcerations occur in 20-30% of patients over age 65. In addition to esophageal erosion and ulceration, prolonged exposure of the esophageal mucosa to stomach acid can lead to a condition known as Barrett's Esophagus. Barrett's Esophagus is an esophageal disorder that is characterized by replacement of normal squamous epithelium with abnormal columnar epithelium. This change in tissue structure is clinically important not only as an indication of severe reflux, but as an indication of cancer.

It is understood that GERD is synonymous with GERD (gastro-oesophageal reflux disease).

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species.

As used herein, treating and treatment refer to a reduction in at least one symptom associated with a gastointestinal motility disorder. For example, the gastrointestinal motility disorder can be GERD and a reduction in heartburn can be realized. In another embodiment, the gastrointestinal motility disorder can be GERD and the subject can experience a reduction in any one or more of the symptoms of dysphagia, odynophagia, hemorrhage, water brash, esophageal erosion, esophageal obstruction and respiratory manifestations such as asthma, recurrent pneumonia, coughing, intermittent wheezing, earache, hoarseness, laryngitis and pharyngitis.

As used herein, increasing esophageal motility refers to increasing peristaltic waves and/or LES pressure.

The invention relates to a method of treating a gastrointestinal motility disorder in a subject in need of treatment comprising coadministering to said subject a first amount of a compound having 5-HT₃ receptor agonist activity or a pharmaceutically acceptable salt, hydrate or solvate thereof and a second amount of at least one gastric acid suppressing agent, wherein the first and second amounts together comprise a therapeutically effective amount. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, hydrate or solvate thereof.

As used herein, therapeutically effective amount refers to an amount sufficient to elicit the desired biological response. In the present invention, the desired biological response is a reduction (complete or partial) of at least one symptom associated with the gastrointestional motility disorder being treated, for example, GERD. As with any treatment, particularly treatment of a multi-symptom disorder, for example, GERD, it is advantageous to treat as many disorder-related symptoms which the subject experiences.

A therapeutically effective amount also refers to an amount sufficient to increase esophageal motility.

A therapeutically effective amount can be achieved in the methods or compositions of the invention by codaministering a first amount of a compound having 5-HT₃ receptor agonist activity or a pharmaceutically acceptable salt, hydrate or solvate thereof and a second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof). A therapeutically effect amount to increase esophageal motility can be achieved by administering a compound having 5-HT₃ receptor agonist activity or a pharmaceutically acceptable salt, hydrate or solvate thereof.

In one embodiment, the compound having 5-HT₃ receptor agonist activity and gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) are each administered in a therapeutically effective amount (i.e., each in an amount which would be therapeutically effective if administered alone). In another embodiment, the compound having 5-HT₃ receptor agonist activity and gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) are each administered in an amount which alone does not provide a therapeutic effect (a sub-therapeutic dose). In yet another embodiment, the compound having 5-HT₃ receptor agonist activity can be administered in a therapeutically effective amount, while the gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) is administered in a sub-therapeutic dose. In still another embodiment, the compound having 5-HT₃ receptor agonist activity can be administered in a sub-therapeutic dose, while the gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) is administered in a therapeutically effective amount.

In certain embodiments, coadministration of a first amount of a compound having 5-HT₃ receptor agonist activity or a pharmaceutically acceptable salt, hydrate or solvate thereof and a second amount of at least one gastric acid suppressing agent such as an H₂ receptor antagonist or a pharmaceutically acceptable salt, hydrate or solvate thereof can result in an enhanced or synergistic therapeutic effect, wherein the combined effect is greater than the additive effect resulting from separate administration of the first amount of the compound having 5-HT₃ receptor agonist activity or a pharmaceutically acceptable salt, hydrate or solvate thereof or the second amount of the gastric acid suppressing agent such as an H2 receptor antagonist or a pharmaceutically acceptable salt, hydrate or solvate thereof.

An advantage of the synergistic effect of the combination therapy is the ability to use less of each agent than is needed when each is administered alone. As such, undesirable side effects associated with the agents are reduced (partially or completely). A reduction in side effects can result in increased patient compliance over current treatments.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

In a particular embodiment, the compounds having 5-HT₃ receptor agonist activity are thieno[3,2-b]pyridine derivatives such as those described in U.S. Pat. No. 5,352,685, the entire content of which is incorporated herein by reference.

In a specific embodiment, the compounds having 5-HT₃ receptor agonist activity are represented by Formula I: 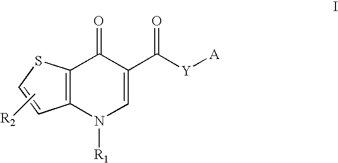
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 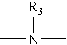
   wherein R₃ represents hydrogen or a C₁-C₆ alkyl group; and
A is represented by 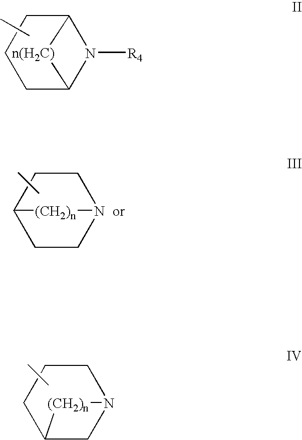
   wherein:
n is an integer from 1 to about 4;
R₄ represents hydrogen, a C₁-C₆ alkyl group, a C₃-C₈ cycloalkyl group or a C₇-C₁₈ aralkyl group;
   or a pharmaceutically acceptable salt, solvate, hydrate or N-oxide derivative thereof.

In one embodiment, the compounds represented by Formula I can be N-oxide derivatives.

In another embodiment of Formula I, Y represents —O— or 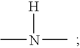
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 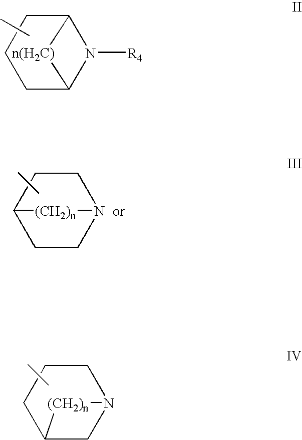
   wherein:
n is 2 or 3;
R₄represents a C₁-C₆ alkyl group.

In a particular embodiment, the compounds having 5-HT₃ receptor agonist activity are represented by Formula I, wherein R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

In a particularly preferred embodiment, the compound having 5-HT₃ receptor agonist activity is represented by structural Formula V: 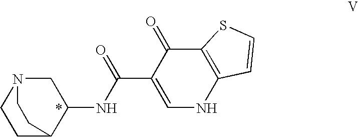
or a pharmaceutically acceptable salt, solvate or hydrate thereof.

In a particular embodiment, the compound represented by Formula I is an N-oxide derivative.

In a particularly preferred embodiment, the compound of Formula V has the (R) configuration at the chiral carbon atom which is designated with an asterisk (*). The chemical name of the compound set forth in Formula V having the (R) configuration at the designated chiral carbon is: (R)—N-1-azabicyclo[2.2.2]oct-3-yl-4,7-dihydro-7-oxothieno[3,2-b]pyridine-6-carboxamide. When the compound is in the form of the monohydrochloride, it is known as MKC 733 (CAS Number: 194093-42-0).

In a particular embodiment, the proton pump inhibitor is selected from the group consisting of esomeprazole, omeprazole, lansoprazole, rabeprazole and pantoprazole.

In a further embodiment, the compound having 5-HT₃ agonist activity is (R)—N-1-azabicyclo[2.2.2]oct-3-yl-4,7-dihydro-7-oxothieno[3,2-b]pyridine-6-carboxamide and the proton pump inhibitor is selected from the group consisting of esomeprazole, omeprazole, lansoprazole, rabeprazole and pantoprazole. In another embodiment, the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide and the proton pump inhibitor is selected from the group consisting of esomeprazole, omeprazole, lansoprazole, rabeprazole and pantoprazole.

In a particular embodiment, the acid pump antagonist is selected from the group consisting of soraprazan, AZD0865, YH1885 and CS-526.

In a further embodiment, the compound having 5-HT₃ agonist activity is (R)—N-1-azabicyclo[2.2.2]oct-3-yl-4,7-dihydro-7-oxothieno[3,2-b]pyridine-6-carboxamide and the acid pump antagonist is selected from the group consisting of soraprazan, AZD0865, YH1885 and CS-526. In another embodiment, the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide and the acid pump antagonist is selected from the group consisting of soraprazan, AZD0865, YH1885 and CS-526.

In another embodiment, the H₂ receptor antagonist is selected from the group consisting of nizatidine, ranitidine, famotidine, roxatidine and cimetidine.

In a further embodiment, the compound having 5-HT₃ agonist activity is (R)—N-1-azabicyclo[2.2.2]oct-3-yl-4,7-dihydro-7-oxothieno[3,2-b]pyridine-6-carboxamide and the H₂ receptor antagonist is selected from the group consisting of nizatidine, ranitidine, famotidine, roxatidine and cimetidine. In yet another embodiment, the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide and the H₂ receptor antagonist is selected from the group consisting of nizatidine, ranitidine, famotidine, roxatidine and cimetidine

In one embodiment, the gastrointestinal motility disorder is GERD. In a particular embodiment, the GERD is nocturnal GERD.

In another embodiment, the gastrointestinal motility disorder is gastroparesis.

In another embodiment, the compounds having 5-HT₃ receptor agonist activity are condensed thiazole derivatives such as those described in U.S. Pat. No. 5,565,479, the entire content of which is incorporated herein by reference.

In a particular embodiment, the compounds having 5-HT₃ receptor agonist activity are represented by Formula VI or a pharmaceutically acceptable salt, solvate or hydrate thereof: 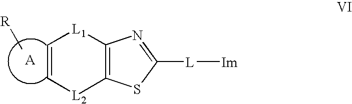
wherein:
R represents hydrogen, halogen, hydroxyl, a C₁-C₆ alkoxy group, carboxy, a C₁-C₆ alkoxycarbonyl group, nitro, amino, cyano or protected hydroxyl; 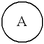
   is a phenyl ring or a naphthalene ring;
L is a direct bond or a C₁-C₆ alkylene group;
L₁ and L₂ are defined so that one is a direct bond and the other is:
   a) a C₁-C₆ alkylene group optionally containing an interrupting oxygen or sulfur atom therein;
   b) an oxygen atom or sulfur atom; or
   c) a C₁-C₆ alkenylene group;
Im represents a group having the formula: 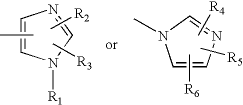
   wherein:
R₁-R₆ are the same or different each representing hydrogen or a C₁-C₆ alkyl group.

In a further embodiment, the compound according to Formula VI, 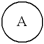
is a phenyl ring, L₁ is a direct bond and L₂ is an alkylene group or alkenylene group.

In a particularly preferred embodiment, the compound having 5-HT₃ receptor agonist activity is represented by structural Formula VII: 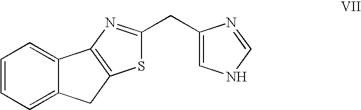
or a pharmaceutically acceptable salt, solvate, or hydrate thereof. This compound is commonly referred to in the art as YM 31636. The chemical name of the compound set forth in the Formula VII is: 2-(1H-imidazol-4-ylmethyl)-8H-indeno[1,2-d]thiazole.

In a particular embodiment, the proton pump inhibitor is selected from the group consisting of esomeprazole, omeprazole, lansoprazole, rabeprazole and pantoprazole.

In a further embodiment, the compound having 5-HT₃ agonist activity is 2-(1H-imidazol-4-ylmethyl)-8H-indeno[1,2-d]thiazole and the proton pump inhibitor is selected from the group consisting of esomeprazole, omeprazole, lansoprazole, rabeprazole and pantoprazole.

In a particular embodiment, the acid pump antagonist is selected from the group consisting of soraprazan, AZD0865, YH1885 and CS-526.

In a further embodiment, the compound having 5-HT₃ agonist activity is 2-(1H-imidazol-4-ylmethyl)-8H-indeno[1,2-d]thiazole and the acid pump antagonist is selected from the group consisting of soraprazan, AZD0865, YH1885 and CS-526.

In another embodiment, the H₂ receptor antagonist is selected from the group consisting of nizatidine, ranitidine, famotidine, roxatidine and cimetidine. In a further embodiment, the compound having 5-HT₃ agonist activity is 2-(1H-imidazol-4-ylmethyl)-8H-indeno[1,2-d]thiazole and the H₂ receptor antagonist is selected from the group consisting of nizatidine, ranitidine, famotidine, roxatidine and cimetidine.

In one embodiment, the gastrointestinal motility disorder is GERD. In a particular embodiment, the GERD is nocturnal GERD.

In another embodiment, the gastrointestinal motility disorder is gastroparesis.

The invention further relates to pharmaceutical compositions for use in therapy or prophylaxis, for example, for the treatment of a gastrointestinal motility disorder in a subject in need of treatment. The pharmaceutical composition comprises a first amount of a compound having 5-HT₃ receptor agonist activity or a pharmaceutically acceptable salt, hydrate or solvate thereof and a second amount of at least one gastric acid suppressing agent. In one embodiment, the gastric acid suppressing agent is selected from the group consisting of a proton pump inhibitor, an H₂ receptor antagonist and a pharmaceutically acceptable salt, hydrate or solvate thereof. In another embodiment, the gastric acid suppressing agent is an acid pump antagonist or a pharmaceutically acceptable salt, hydrate or solvate thereof. The pharmaceutical compositions of the present invention can optionally contain a pharmaceutically acceptable carrier. The first amount of a compound having 5-HT₃ receptor agonist activity or a pharmaceutically acceptable salt, hydrate or solvate thereof and the second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) can together comprise a therapeutically effective amount.

In one embodiment, the gastrointestinal motility disorder treated with the pharmaceutical composition is GERD. In a particular embodiment, the GERD is nocturnal GERD.

In another embodiment, the gastrointestinal motility disorder treated with the pharmaceutical composition is gastroparesis.

Pharmaceutically acceptable carrier, includes pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers can be aqueous or non-aqueous solvents. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

Modes of Administration

The compounds for use in the methods or compositions of the invention can be formulated for oral, transdermal, sublingual, buccal, parenteral, rectal, intranasal, intrabronchial or intrapulmonary administration. For oral administration the compounds can be of the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the methods or compositions of the invention can be in the form of tablets or lozenges formulated in a conventional manner.

For parenteral administration, the compounds for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For rectal administration, the compounds for use in the methods or compositions of the invention can be in the form of suppositories.

For sublingual administration, tablets can be formulated in conventional manner.

For intranasal, intrabronchial or intrapulmonary administration, conventional formulations can be employed.

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

Additional dosage forms suitable for use in the methods or compositions of the invention include dosage forms as described in U.S. Pat. No. 6,340,475, U.S. Pat. No. 6,488,962, U.S. Pat. No. 6,451,808, U.S. Pat. No. 6,340,475, U.S. Pat. No. 5,972,389, U.S. Pat. No. 5,582,837, and U.S. Pat. No. 5,007,790. Additional dosage forms include those described in U.S. patent application No. 20030147952, U.S. patent application No. 20030104062, U.S. patent application No. 20030104053, U.S. patent application No. 20030044466, U.S. patent application No. 20030039688, and U.S. patent application No. 20020051820. Additional dosage forms of this invention also include dosage forms as described in PCT patent application WO 03/35041, PCT patent application WO 03/35040, PCT patent application WO 03/35029, PCT patent application WO 03/35177, PCT patent application WO 03/35039, PCT patent application WO 02/96404, PCT patent application WO 02/32416, PCT patent application WO 01/97783, PCT patent application WO 01/56544, PCT patent application WO 01/32217, PCT patent application WO 98/55107, PCT patent application WO 98/11879, PCT patent application WO 97/47285, PCT patent application WO 93/18755, and PCT patent application WO 90/11757.

In one embodiment, the dosage forms of the present invention include pharmaceutical tablets for oral administration as described in U.S. patent application No. 20030104053. The dosage forms of this invention include dosage forms in which the same drug is used in both the immediate-release and the prolonged-release portions as well as those in which one drug is formulated for immediate release and another drug, different from the first, for prolonged release. This invention is particularly directed to dosage forms in which the immediate-release drug is at most sparingly soluble in water, i.e., either sparingly soluble or insoluble in water, while the prolonged-release drug can be of any level of solubility.

More particularly, the prolonged-release portion of the dosage form can be a dosage form that delivers drug to the digestive system continuously over a period of time of at least an hour and preferably several hours and the drug is formulated as described in U.S. patent application No. 20030104053. In said embodiment, the immediate-release portion of the dosage form is either a coating applied or deposited over the entire surface of a unitary prolonged-release core, or a single layer of a tablet constructed in two or more layers, one of the other layers of which is the prolonged-released portion and is formulated as described in U.S. patent application No. 20030104053.

In another embodiment of the invention, the supporting matrix in controlled-release tablets or controlled release portions of tablets is a material that swells upon contact with gastric fluid to a size that is large enough to promote retention in the stomach while the subject is in the digestive state, which is also referred to as the postprandial or “fed” mode. This is one of two modes of activity of the stomach that differ by their distinctive patterns of gastroduodenal motor activity. The “fed” mode is induced by food ingestion and begins with a rapid and profound change in the motor pattern of the upper gastrointestinal (GI) tract. The change consists of a reduction in the amplitude of the contractions that the stomach undergoes and a reduction in the pyloric opening to a partially closed state. The result is a sieving process that allows liquids and small particles to pass through the partially open pylorus while indigestible particles that are larger than the pylorus are retropelled and retained in the stomach. This process causes the stomach to retain particles that are greater than about 1 cm in size for about 4 to 6 hours. The controlled-release matrix in these embodiments of the invention is therefore selected as one that swells to a size large enough to be retropelled and thereby retained in the stomach, causing the prolonged release of the drug to occur in the stomach rather than in the intestines. Disclosures of oral dosage forms that swell to sizes that will prolong the residence time in the stomach are found in U.S. Pat. No. 6,448,962, U.S. Pat. No. 6,340,475, U.S. Pat. No. 5,007,790, U.S. Pat. No. 5,582,837, U.S. Pat. No. 5,972,389, PCT patent application WO 98/55107, U.S. patent application No. 20010018707, U.S. patent application No. 20020051820, U.S. patent application No. 20030029688, U.S. patent application No. 20030044466, U.S. patent application No. 20030104062, U.S. patent application No. 20030147952, U.S. patent application No. 20030104053, and PCT patent application WO 96/26718. In particular, gastric retained dosage formulations for specific drugs have also been described, for example a gastric retained dosage formulation for gabapentin is disclosed in PCT patent application WO 03/035040.

Coadministration

When the methods of the invention include coadministration, coadministration refers to administration of a first amount of a compound having 5-HT₃ receptor agonist activity or a pharmaceutically acceptable salt, hydrate or solvate thereof and a second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof), wherein the first and second amounts together comprise a therapeutically effective amount to treat a gastrointestinal motility disorder or for increasing esophageal motility in a subject in need of treatment. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When coadministration involves the separate administration of the first amount of the compound having 5-HT₃ receptor agonist activity of a pharmaceutically acceptable salt, hydrate or solvate thereof and a second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) the compounds are administered sufficiently close in time to have the desired therapeutic effect. For example, the period of time between each administration, which can result in the desired therapeutic effect, can range from minutes to hours and can be determined taking into account the properties of each compound such as potency, solubility, bioavailability, plasma half-life and kinetic profile. For example, the compound having 5-HT₃ receptor agonist activity and at least one gastric acid suppressing agent (e.g., a proton pump inhibitor, an H2 receptor antagonist or a pharmaceutically acceptable salt, hydrate or solvate thereof; or an acid pump antagonist or pharmaceutically acceptable salt, hydrate or solvate thereof) can be administered in any order within about 24 hours of each other, within about 16 hours of each other, within about 8 hours of each other, within about 4 hours of each other, within about 1 hour of each other or within about 30 minutes of each other.

In a particular embodiment when the coadministration comprises oral administration of a first amount of a compound having 5-HT₃ receptor agonist activity and a second amount of a gastric acid suppressing agent in a single composition, it is preferred that the gastric acid suppressing agent releases first followed by the compound having 5-HT₃ receptor agonist activity. Release of the agents can occur in the stomach, duodenum or both. For example, a single oral composition can be formulated such that the compound having 5-HT₃ receptor agonist activity and the gastric acid suppressing agent release in the stomach, duodenum or both. In addition, the composition can be formulated to release the gastric acid suppressing agent first, followed by the compound having 5-HT₃ receptor agonist activity. Staggered release of agents can be accomplished in single composition using any suitable formulation technique such as those described above. For example, a variety of coating thicknesses and/or different coating agents can provide staggered release of agents from a single composition, and release at a desired location in the upper GI tract. In a particular embodiment, a single composition having two portions can be prepared. Portion 1 can be the gastric acid suppressing agent and portion 2 can be the compound having 5-HT₃ receptor agonist activity. As a first step following administration, the single composition separates into the individual portions. Portion 1 can begin to release immediately and portion 2 can be formulated to release later, for example, about 3 or more hours later.

When the coadministration comprises administration of a compound having 5-HT₃ receptor agonist activity and a gastric acid suppressing agent as separate compositions, either at the same time or sequentially, the separate compositions can be formulated to achieve the desired release profile. For example, the separate compositions can be formulated to release primarily in the duodenum rather than in the acidic environment of the stomach. In addition, the separate compositions can be formulated such that the gastric acid suppressing agent releases first followed by the 5-HT₃ receptor agonist, taking into consideration the amount of time between administration of the separate compositions. A variety of formulation techniques such as gastric retention techniques, coating techniques and the use of suitable excipients and/or carriers can be utilized to achieve the desired release.

An additional therapeutic agent can be used in the method of treating a gastrointestinal motility disorder, in the method of increasing esophageal motility and in compositions of the invention described herein. Additional therapeutic agents suitable for use in the method of treating a gastrointestinal motility disorder, in the method of increasing esophageal motility and in compositions of the invention can be, but are not limited to, antacids, for example, TUMS® and ROLAIDS®. Generally, the additional therapeutic agent will be one that is useful for treating the disorder of interest. Preferably, the additional therapeutic agent does not diminish the effects of the therapy and/or potentiates the effects of the primary administration.

Dosing

The therapeutically effective amount of a first amount of a compound having 5-HT₃ receptor agonist activity and a second amount of at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) in combination will depend on the age, sex and weight of the patient, the current medical condition of the patient and the nature of the gastrointestinal motility disorder being treated. The skilled artisan will be able to determine appropriate dosages depending on these and other factors.

As used herein, continuous dosing refers to the chronic administration of a selected active agent.

As used herein, as-needed dosing, also known as “pro re nata” “prn” dosing, and “on demand” dosing or administration is meant the administration of a therapeutically effective dose of the compound(s) at some time prior to commencement of an activity wherein suppression of an gastrointestinal motility disorder would be desirable. Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation. For example, the combination therapy can be administered about one hour before sleep to treat nocturnal GERD.

In a particularly preferred embodiment, the treatment of nocturnal GERD comprises administration of the gastric acid suppressing agent about 30 minutes before the last meal of the day (e.g., dinner) followed by administration of the compound having 5-HT₃ receptor agonist activity around bedtime. As described above, this treatment regimen can also be achieved with administration of a single composition formulated to provide a release profile similar to that achieved with the staggered administrations or with administration of separate agents at the same time or close in time but each formulated to achieve the staggered release.

In a particular embodiment, drug administration or dosing is on an as-needed basis, and does not involve chronic drug administration. With an immediate release dosage form, as-needed administration can involve drug administration immediately prior to commencement of an activity wherein suppression of the symptoms of the gastrointestinal motility disorder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

A suitable dose per day for each of the compound having 5-HT₃ receptor agonist activity or the gastric acid suppressing agent (e.g., a proton pump inhibitor, an H₂ receptor antagonist or a pharmaceutically acceptable salt, hydrate or solvate thereof, or an acid pump antagonist or pharmaceutically acceptable salt, hydrate or solvate thereof) for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Other suitable doses per day for each of the compound having 5-HT₃ receptor agonist activity or the gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) for administration include doses of about or greater than 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg (0.5 mg), about 1 mg, about 1.25 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg.

In a particular embodiment, a suitable dose of 5-HT₃ receptor agonist can be in the range of from about 0.1 mg to about 100 mg per day, such as from about 0.5 mg to about 50 mg, for example, from about 1 mg to about 25 mg per day. The dose can be administered in a single dosage or in multiple dosages, for example from 1 to 4 or more times per day. When multiple dosages are used, the amount of each dosage can be the same or different.

In a particular embodiment, a suitable dose of the proton pump inhibitor can be in the range of from about 0.20 mg to about 2000 mg per day, such as from about 1 mg to about 1000 mg, for example, from about 5 mg to about 500 mg, such as about 10 mg to about 250 mg per day. The dose can be administered in a single dosage or in multiple dosages, for example from 1 to 4 or more times per day. When multiple dosages are used, the amount of each dosage can be the same or different.

In a particular embodiment, a suitable dose of the H₂ receptor antagonist can be in the range of from about 0.20 mg to about 4000 mg per day, such as from about 1 mg to about 4000 mg, for example, from about 5 mg to about 3000 mg, such as about 10 mg to about 2400 mg per day. The dose can be administered in a single dosage or in multiple dosages, for example from 1 to 4 or more times per day. When multiple dosages are used, the amount of each dosage can be the same or different.

In a particular embodiment, a suitable dose of the acid pump antagonist can be in the range of from about 0.02 mg to about 20 g per day, such as from about 0.10 mg to about 10 g per day, for example, from about 0.2 mg to about 5 g per day, such as from about 0.40 mg to about 2.5 g per day, for example, from about 0.80 mg to about 1.25 g per day.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. Suitable amounts for use in preparation of a unit dosage form are described above for both the 5-HT₃ receptor agonist and gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof). The unit dosage form can be for a single daily dose or one of multiple daily doses (e.g., about 1 to 4 or more times per day). When multiple daily doses are used, the unit dosage form can be the same or different for each dose.

The invention further includes a kit for treating a gastrointestinal motility disorder or for increasing esophageal motility. The kit comprises a compound having 5-HT₃ receptor agonist activity or a pharmaceutically acceptable salt, hydrate or solvate thereof and instructions for use with at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof), according to the method of the invention and optionally a device for administering the compounds of the invention. In a particular embodiment, the compound having 5-HT₃ receptor agonist activity is present in the kit in a sub-therapeutic dose. In another embodiment, the instructions direct administration of the gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) in a sub-therapeutic dose.

The invention further includes a kit for treating a gastrointestinal motility disorder or for increasing. The kit comprises at least one gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) and instructions for use with a compound having 5-HT₃ receptor agonist activity or a pharmaceutically acceptable salt, hydrate or solvate thereof, according to the method of the invention and optionally a device for administering the compounds of the invention. In a particular embodiment, the gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) is present in the kit in a sub-therapeutic dose. In another embodiment, the instructions direct administration of the compound having 5-HT₃ receptor agonist activity in a sub-therapeutic dose.

The invention further includes a kit for treating a gastrointestinal motility disorder or for increasing esophageal motility. The kit comprises a first compound having 5-HT₃ receptor agonist activity or a pharmaceutically acceptable salt, hydrate or solvate thereof, a second compound which is a gastric acid suppressing agent (e.g., a proton pump inhibitor, an H₂ receptor antagonist or a pharmaceutically acceptable salt, hydrate or solvate thereof; or an acid pump antagonist or pharmaceutically acceptable salt, hydrate or solvate thereof) and instructions for administering the first and second compounds, according to the method of the invention and optionally a device for administering the compounds of the invention. In a particular embodiment, at least one of the first or second compound is present in the kit in a sub-therapeutic dose.

Compounds can be in separate dosage forms or combined in a single dosage form. In other embodiments of the kits, the instructional insert further includes instructions for administration with an additional therapeutic agent as described herein.

It is understood that in practicing the method or using a kit of the present invention that administration encompasses administration by different individuals (e.g., the subject, physicians or other medical professionals) administering the same or different compounds.

As used herein, the term pharmaceutically acceptable salt refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

The active compounds disclosed can be prepared in the form of their hydrates, such as hemihydrate, monohydrate, dihydrate, trihydrate, tetrahydrate and the like and as solvates.

It is understood that suitable compounds having 5-HT₃ receptor agonists activity, proton pump inhibitors and H₂ receptor antagonists can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

An “aliphatic group” is non-aromatic, consists solely of carbon and hydrogen and can optionally contain one or more units of unsaturation, e.g., double and/or triple bonds and/or one or more suitable substituents. An aliphatic group can be straight chained, branched or cyclic. When straight chained or branched, an aliphatic group typically contains between about 1 and about 12 carbon atoms, more typically between about 1 and about 6 carbon atoms. When cyclic, an aliphatic group typically contains between about 3 and about 10 carbon atoms, more typically between about 3 and about 8 carbon atoms, e.g., a cyclopropyl group, cyclohexyl group, cyclooctyl group etc. Aliphatic groups can be alkyl groups (i.e., completely saturated aliphatic groups, e.g., a C₁-C₆ alkyl group, such as a methyl group, propyl group, hexyl group, etc.), alkenyl groups (i.e., aliphatic groups having one or more carbon-carbon double bonds, e.g., C₂-C₆ alkenyl group, such as a vinyl group, butenyl group, hexenyl group etc.) or alkynyl groups (i.e., aliphatic groups having one or more carbon-carbon triple bonds, e.g., a C₂-C₆ alkynyl group, such as an ethynyl group, butynyl group, hexenyl group, etc.). Aliphatic groups can optionally be substituted with a designated number of substituents, as described herein.

Alkylene group as used herein refers to the triatomic group having one carbon atom and two attached hydrogens (—CH₂— or ═CH₂) groups such as C₁-C₆ alkylene, for example, methylene, ethylene, methylmethylene, trimethylene, 1-methylethylene etc.

Alkenylene group as used herein refers to the diatomic group having one carbon atom and one attached hydrogen. Suitable alkenylene groups include C₂-C₆ alkenylene groups such as vinylene, propenylene, 1-methylvinylene, etc.

An “aromatic group” (also referred to as an “aryl group”) as used herein includes carbocyclic aromatic groups, heterocyclic aromatic groups (also referred to as “heteroaryl”) and fused polycyclic aromatic ring systems as defined herein which can be optionally substituted with a suitable substituent.

A “carbocyclic aromatic group” is an aromatic ring of 5 to 14 carbons atoms, and includes a carbocyclic aromatic group fused with a 5- or 6-membered cycloalkyl group such as indan. Examples of carbocyclic aromatic groups include, but are not limited to, phenyl, naphthyl, e.g., 1-naphthyl and 2-naphthyl; anthracenyl, e.g., 1-anthracenyl, 2-anthracenyl; phenanthrenyl; fluorenonyl, e.g., 9-fluorenonyl, indanyl and the like. A carbocyclic aromatic group is optionally substituted with a designated number of substituents, described below.

A “heterocyclic aromatic group” (or “heteroaryl”) is a monocyclic, bicyclic or tricyclic aromatic ring of 5- to 14-ring atoms of carbon and from one to four heteroatoms selected from O, N, or S. Examples of heteroaryl include, but are not limited to pyridyl, e.g., 2-pyridyl (also referred to as α-pyridyl), 3-pyridyl (also referred to as β-pyridyl) and 4-pyridyl (also referred to as γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like. Heterocyclic aromatic (or heteroaryl) as defined above can be optionally substituted with a designated number of substituents, as described below for aromatic groups.

A “fused polycyclic aromatic” ring system is a carbocyclic aromatic group or heteroaryl fused with one or more other heteroaryl or nonaromatic heterocyclic ring. Examples include, quinolinyl and isoquinolinyl, e.g., 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g., 2-benzofuranyl and 3-benzofuranyl; dibenzofuranyl, e.g., 2,3-dihydrobenzofuranyl; dibenzothiophenyl; benzothienyl, e.g., 2-benzothienyl and 3-benzothienyl; indolyl, e.g., 2-indolyl and 3-indolyl; benzothiazolyl, e.g., 2-benzothiazolyl; benzooxazolyl, e.g., 2-benzooxazolyl; benzimidazolyl, e.g., 2-benzoimidazolyl; isoindolyl, e.g., 1-isoindolyl and 3-isoindolyl; benzotriazolyl; purinyl; thianaphthenyl and the like. Fused polycyclic aromatic ring systems can optionally be substituted with a designated number of substituents, as described herein.

An “aralkyl group” (arylalkyl) is an alkyl group substituted with an aromatic group, preferably a phenyl group. A preferred aralkyl group is a benzyl group. Suitable aromatic groups are described herein and suitable alkyl groups are described herein. An aralkyl group can optionally be substituted, and suitable substituents for an aralkyl group (substituted on the aryl, alkyl or both moieties) are described herein.

As used herein, many moieties or groups are referred to as being either “substituted or unsubstituted”. When a moiety is referred to as substituted, it denotes that any portion of the moiety that is known to one skilled in the art as being available for substitution can be substituted. For example, the substitutable group can be a hydrogen atom which is replaced with a group other than hydrogen (i.e., a substituent group). Multiple substituent groups can be present. When multiple substituents are present, the substituents can be the same or different and substitution can be at any of the substitutable sites on the group or moiety. Such means for substitution are well-known in the art. For purposes of exemplification, which should not be construed as limiting the scope of this invention, some examples of groups that are substituents are: alkyl groups (e.g., C₁-C₆ alkyl groups) which can also be substituted, such as CF₃), alkoxy groups (e.g., C₁-C₆ alkoxy, such as a methoxy group, propoxy group, hexyloxy group etc.) which can be substituted, such as OCF₃), a halogen or halo group (F, Cl, Br, I), hydroxy, nitro, thio (also referred to as mercapto), akylthio (e.g., C₁-C₆ alkylthio), oxo, —CN, —COH, —COOH, amino, N-alkylamino (e.g., C₁-C₆ alkylamino) or N,N-dialkylamino (in which the alkyl groups can also be substituted), esters (—C(O)—OR, where R can be a group such as alkyl, aryl, etc., which can be substituted), aryl (most preferred is phenyl, which can be substituted) and arylalkyl (which can be substituted).

N-oxide refers a functionality wherein an oxygen atom is bonded to the nitrogen of a tertiary amine.

Protected hydroxyl refers to a hydroxyl group in which the hydrogen atom of the hydroxy group has been replaced with a suitable hydroxy protecting group. Suitable hydroxy protecting groups include but are not limited to, for example, benzyl, tert-butyl, acetyl, trifluoroacetyl, benzoyl and benzyloxycarbonyl.

Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When compounds of the present invention contain one chiral center, the compounds exist in two enantiomeric forms and the present invention includes either or both enantiomers and mixtures of enantiomers, such as the specific 50:50 mixture referred to as a racemic mixture. The enantiomers can be resolved by methods known to those skilled in the art, for example by formation of diastereoisomeric salts which may be separated, for example, by crystallization (See, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least about 99% enantiomeric excess.

When a compound of the present invention has two or more chiral carbons, it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

Pharmacological Methods

The efficacy of the combination therapy can be assessed through monitoring of the patient's symptoms. For example, an improvement in symptoms such as, hoarseness, cough, heartburn, asthma and overall quality of life can be assessed without the need for invasive testing.

In addition, patients receiving the combination therapy can be subjected to gastroesophageal testing, for example, esophageal manometry followed by ambulatory gastroesophageal pH monitoring. This type of gastoesophageal testing can be conducted according to established protocols such as those found in Fackler et al., Gastroenterology 122(3): 625-632 (2002).

Esophageal Manometry

Briefly, esophageal manometry is used to locate the LES of all study participants using the station pull-through technique. LES pressure and location are recorded by a computerized motility system such as Synectics Gastrosoft Polygram, Milwaukee, Wis.

Ambulatory Gastroesophageal pH Monitoring

Twenty-four hour pH level monitoring is then conducted in all study participants. Monitoring is performed with 2.1 mm monocrystalline pH catheters with 2 antimony electrodes separated by 15 cm (Medtronic Functional Diagnostics Zinetics, Inc., Salt lake City, Utah). The reference electrode is internalized. The pH electrodes are calibrated at 37° C. in buffer solutions of pH 7 and pH 1 (Fisher Scientific, Fairlawn, N.J.) before each study. After calibration, the pH probe apparatus is passed nasally and positioned such that the distal electrode is in the gastric findus, 10 cm below the proximal border of the lower esophageal sphincter. The probe apparatus is secured to the nose and cheek to prevent dislodgment. The pH electrodes are connected to a portable digital data recorder (Digitrapper Mark III Gold; Synectics) worn around the waist, which stores pH data samples every 4 seconds for up to 24 hours. Patients then return home with instructions to keep a diary recording meal times, time of lying down for sleep, and time of rising in the morning. Patients are encouraged to perform their normal daily activities, consume their customary diet without restrictions, and avoid sleeping for short periods during the day. They return the following day after a minimum of 18 hours to have their probes removed and their diaries reviewed.

Additional pH monitoring following onset of combination therapy is conducted at predetermined time points and the data compared and analyzed to determine the effectiveness among combination therapies and the effectiveness of combination therapy as compared to monotherapy with the components of the combination.

Assessment of Suppression of Gastric Acid Following Histamine Stimulation

The ability of the combination therapy to suppress gastric acid can be assessed using the fundic pouch dog model. More specifically, following starvation overnight a dog is subjected to sterile ventrotomy under anesthesia using sodium pentobarbital (about 30 mg/kg, i.v.) and a fistula is attached to a part of the corpus ventriculi. After a two week recovery period, the dog is fixed to the Pavlov's stand, and gastric juice is collected every 15 minutes for about 4 hours under histamine stimulation (about 0.2 mg/kg/hr). A volume of each collected juice is recorded and the juice is titrated with 0.01 N NaOH using pH automatic measuring apparatus. The amount of gastric juice secreted in calculated as mEq/4 hr. The combination therapy is then orally administered about one hour before histamine administration and gastric juice is collected and analyzed as described for the control group. Comparison of the amount of gastric acid secreted for the Control and Treated Groups is conducted to assess the ability of the combination therapy to suppress gastric acid secretion.

Assessment of Suppression of Gastric Acid Following Tetragastrin Stimulation

The method described above using histamine as the stimulating agent is conducted to assess the ability of the combination therapy to suppress gastric acid secretion but using tetragastrin as the stimulating agent (2 μg/kg/hr).

Acid Clearance and pH Monitoring

pH monitoring is also conducted in animals. Suitable examples of experimental studies can be found in: Gawad, K. A., et al., Ambulatory long-term pH monitoring in pigs, Surg Endosc, (2003); Johnson, S. E. et al., Esophageal Acid Clearance Test in Healthy Dogs, Can. J. Vet. Res. 53(2): 244-7 (1989); and Cicente, Y. et al., Esophageal Acid Clearance: More Volume-dependent Than Motility Dependent in Healthy Piglets, J. Pediatr. Gastroenterol. Nutr. 35(2): 173-9 (2002).

Experimental Methods

Effect of Treatment on Lower Esophageal Sphincter Pressure (LESP), Lower Esophageal pH, Esophageal Motility and Transient Lower Esophageal Relaxation (TLESR)

Experiments to determine the effects of MKC-733, omeprazole or the combination of MKC-733 and omeprazole on LESP, lower esophageal pH, esophageal motility and TLESR, in a feline model of GERD were conducted.

Preparation of Animals:

The cats used in the experiments were fasted overnight and sedated with ketamine (15-20 mg/kg intramuscular injection). A butterfly catheter filled with heparinized sterile saline was placed into the brachial vein and used for supplemental ketamine anesthesia and drug administration.

Methods for Measuring LESP, Lower Esophageal pH, Esophageal Motility and TLESR:

Each animal was fitted with a water-perfused sleeve catheter (Andorfer Inc, Greendale, Wis.) attached via pressure transducers to a minimally compliant hydrolytic pump. The sleeve was positioned within the LES with the tip placed into the stomach. The total distance between recording site 0 (tip in the stomach) and recording site 2 was 4 cm. This 4 cm region was referred to as site 1 and the pressure was simultaneously recorded along this region. The remaining recording sites (3, 4 and 5) were 2 cm apart with site 5 placed at about 6 cm from the top of the sleeve. The LES was located by moving the sleeve until the tip (site 0) showed a rapid drop in pressure to about 0 mm Hg and the proximal site 1 maintained high tonic pressure (about 54±3 mm Hg). Throughout the experiment, the output from the pressure transducers was manometrically recorded using the PowerLab Chart 5 data acquisition program (ADInstruments, Colorado Springs, Colo.) on a computer using a Windows XP operating system.

An Orion II pH probe (Medical Measurements Systems), running along with the manometric catheter, was positioned with one pH measuring site in the stomach and a second pH measuring site in the distal esophagus. pH was monitored and recorded simultaneously with the manometric recordings using a computerized data acquisition system (Medical Measurements Systems).

LESP Measurement

The manometric pressure recording at site 1 of the catheter provided the baseline (at rest) LESP for each animal. The baseline LESP was recorded for each measurement regimen set forth in Experiments 1 and 2 below, and then compared.

Esophageal Motility and TLESR Measurements

The manometric pressure recordings at sites 1-5 of the sleeve catheter were recorded during primary peristalsis induced by three spontaneous dry swallows (SDS) and secondary peristalsis induced by 3 balloon distensions (BD; distension of a balloon catheter 2 cm in diameter for 5 second placed in the mid portion of the esophagus).

Esophageal motility was characterized based on the amplitude of the contractions recorded at sites 2-5 of the catheter in response to three SDS and three BD. The esophageal motility was characterized for each measurement regimen set forth in Experiments 1 and 2.

When the peristaltic wave induced by SDS and BD reaches the LES, there is a relaxation of the LES, referred to as TLESR. The TLESR can be characterized based on the pressure change of the LES induced by SDS and BD at recorded at site 1 of the sleeve catheter and expressed relative to the pressure at site 0 (in the stomach). Attempts to characterize the TLESR in the cat for each measurement regimen set forth in Experiments 1 and 2 were unsuccessful. However, a similar study design in other animals, for example, dogs or ferrets could provide TLESR measurements.

The methodology for recording of distal esophageal peristalsis and LESP is adopted from Blank et al., Am. J. Physiol. 257: G517-G523, 1989; Greenwood et al., Am J. Physiol. 262: G567-G571, 1992; and Greenwood et al., Gastroenterology 106: 624-628, 1994.

pH of the Lower Esophagus

The pH in the lower esophagus was monitored at the same time as the manometric pressure. The pH was recorded for each measurement regimen set forth in Experiments 1 and 2.

Study Design:

All animals were acclimated to the facility for one week prior to testing. Administration of drug and measurements of the LESP, esophageal pH, esophageal peristalsis and TLESR were conducted on sedated animals (15-20 mg/kg ketamine intramuscular injection). Ketamine administration was controlled to maintain sedation but not alter the ability of the cat to swallow. Throughout the experiment the animals were placed on a heating blanket (37° C.) to maintain body temperature.

Five male cats in total were used in the cumulative dose-response experiments below. Each experiment employs the same five cats. Therefore, each animal serves as its own control within experiments and between experiments.

Experiment 1

Following instrumentation, baseline values of LESP, esophageal pH, esophageal peristalsis and TLESR were measured as described above. Immediately following these physiological measurements, vehicle alone was given intravenously (30% polyethylene glycol in phosphate buffered saline). Physiological measurements were repeated during the 0-5 minutes post-injection period to determine vehicle effects, if any. Fifteen minutes later, 1.0 mg/kg MKC-733 in vehicle (same as above) was given intravenously and physiological measurements were again taken. Fifteen minutes later, 10 mg/kg MKC-733 in vehicle (same as above) was given intravenously and physiological measurements were again taken. The animals were then uninstrumented, allowed to recover from anesthesia, and returned to their cages.

Experiment 2

After 3 days of recovery, the animals began a 4-day pretreatment with the PPI, omeprazole, at a dose of 20 mg/kg (propylene glycol vehicle) administered intraperitoneally (i.p.) once a day. The pretreatment ensured inhibition of the H+−K+ATPase of the gastric parietal cells. One hour after the last omeprazole injection, cats were again sedated and instrumented as described above and the dose-response for MKC-733 as described in its entirety for Experiment 1 was repeated.

Data Analysis:

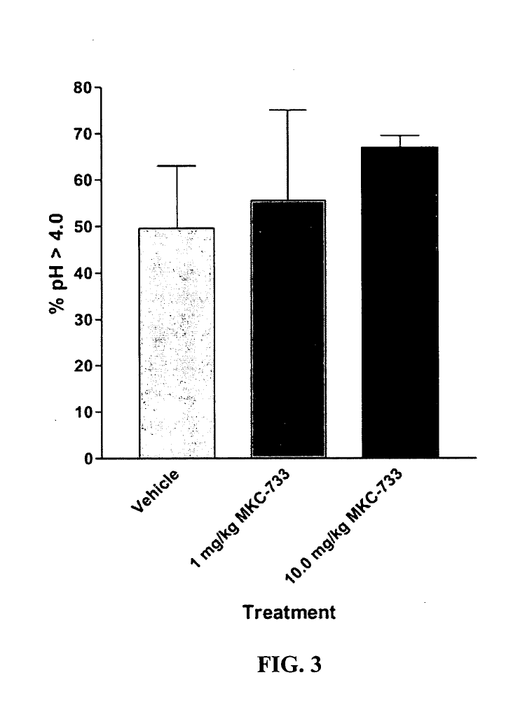

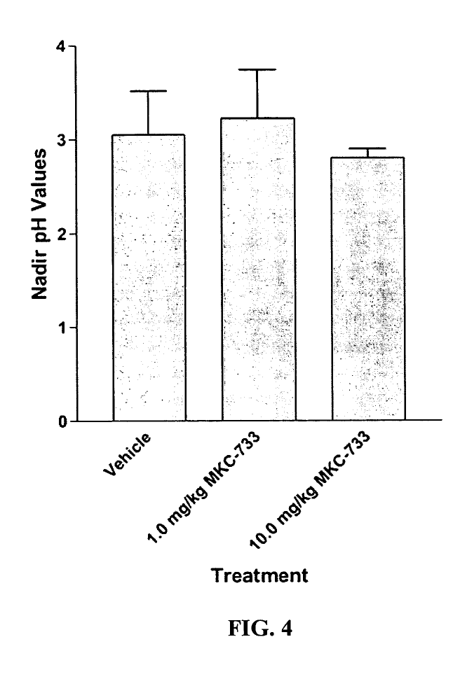

Data is presented as mean ±SEM. LESP and Peristaltic Contraction Amplitude data were normalized to vehicle control values. Significance of LESP treatment effects within and between experiments was evaluated using 2-Way repeated measures ANOVA. In addition to nadir gastroesophageal reflux (GER) pH values (FIG. 4), pH data was also examined within a 2.5 minute duration (initiated at the start of the pH drop due to transient GER caused by spontaneous swallows or esophageal balloon distensions) and was normalized to the percentage of time that pH was below 4.0 during this period (FIG. 3). Significance of treatment effects for pH was evaluated using a nonparametric one-way repeated measures ANOVA (Friedman Test). Additional comparisons were made utilizing paired and unpaired t tests. P<0.05 was considered significant.

Because chronic pretreatment with omeprazole collapsed the pH gradient between the lower esophagus and the stomach, pH data from the animals following this pretreatment was not analyzed. In 2 of the 5 animals, the 10 mg/kg dose was not administered. In 1 of the 3 remaining animals, pH was also not recorded following the 10 mg/kg dose.

Results:

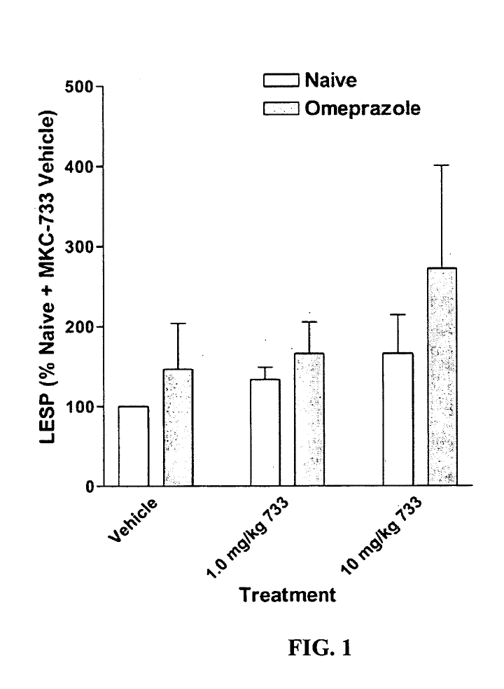

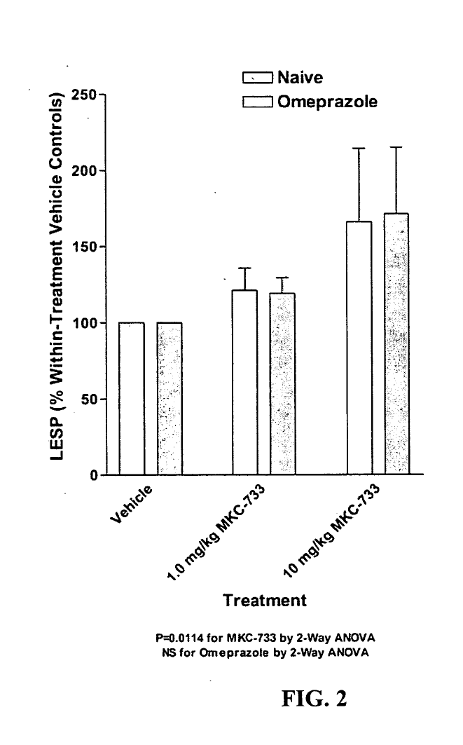

Surprisingly, when the LESP data were normalized for each animal to its naive vehicle control, an enhancement of LESP due to omeprazole pretreatment is apparent with and without treatment with MKC-733 (FIG. 1). Moreover, when the data were normalized to vehicle controls within experiments (naive treatments normalized to naive vehicle, omeprazole treatment normalized to omeprazole vehicle), intravenous administration of MKC-733 led to a statistically significant dose-dependent increase in LESP (P<0.0 114 for MKC-733 dose-response by 2-Way ANOVA) independent of omeprazole pretreatment (FIG. 2).

Surprisingly, intravenous administration of MKC-733 also resulted in a positive trend that appeared dose-dependent in the percentage of time during gastroesophageal (GER) episodes when lower esophageal pH was greater than 4.0 (FIG. 3), even though MKC-733 did not effect nadir pH values during GER at any dose (FIG. 4).

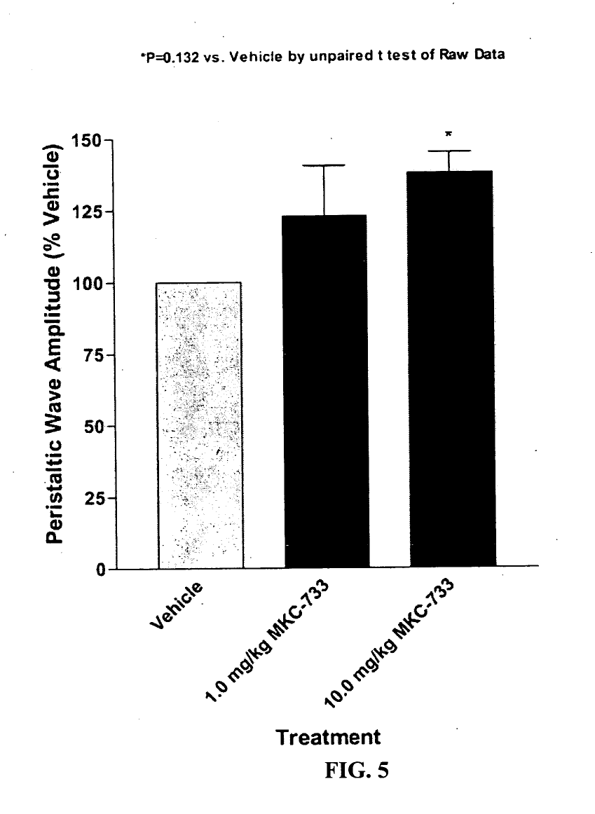

In addition to the above results which all demonstrate a direct effect of MKC-733 on lower esophageal sphincter tone, MKC-733 was also observed to demonstrate a dose-dependent significant enhancement of oral-to-aboral peristaltic contraction amplitude (FIG. 5). There were no significant differences in this effect seen between na\dot{i}ve cats and those pretreated with omeprazole (data not shown).

The above results show that the combination of MKC-733 and an acid suppressing agent can be a suitable treatment for subjects having gastrointestinal motility disorders, such as GERD, particularly nocturnal GERD. For example, the observed increase in LESP and in the period of time that the pH was greater than 4.0 during gastroesophageal reflux, show that the exposure time of the lower esophagus to the damaging effects of the gastric content can be reduced.

In addition, the results show that esophageal motility is increased in animals receiving MKC-733 even absent omeprazole pretreatment. This increased esophageal motility can provide a suitable therapy for the treatment of gastrointestinal motility disorders such as GERD, particularly nocturnal GERD.

While this invention has been particularly shown and described with references to preferred embodiments thereof, it will be understood by those skilled in the art that various changes in form and details may be made therein without departing from the scope of the invention encompassed by the appended claims.

1. A method of treating a gastrointestinal motility disorder in a subject in need of treatment comprising coadministering to said subject:
a) a first amount of a compound having 5-HT₃ receptor agonist activity or a pharmaceutically acceptable salt, hydrate or solvate thereof; and
b) a second amount of at least one gastric acid suppressing agent or a pharmaceutically acceptable salt, hydrate or solvate thereof,
wherein the first and second amounts together comprise a therapeutically effective amount.

2. The method of claim 1, wherein the gastric acid suppressing agent is a proton pump inhibitor, an H₂ receptor antagonist or a pharmaceutically acceptable salt, hydrate or solvate thereof.

3. The method of claim 1, wherein the gastric acid suppressing agent is an acid pump antagonist or a pharmaceutically acceptable salt, hydrate or solvate thereof.

4. The method of claim 1, wherein the gastrointestinal motility disorder is GERD.

5. The method of claim 4, wherein the GERD is nocturnal GERD.

6. The method of claim 1, wherein the gastrointestinal motililty disorder is gastroparesis.

7. The method of claim 1, wherein the subject is a human.

8. The method of claim 1, wherein the compound having 5-HT₃ receptor agonist activity is thieno[3,2-b]pyridine derivative.

9. The method of claim 8, wherein the compound having 5-HT₃ receptor agonist activity is represented by Formula I: 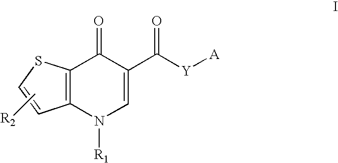
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 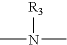
wherein R₃ represents hydrogen or a C₁-C₆ alkyl group; and
A is represented by 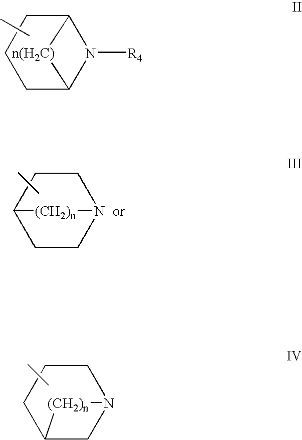
wherein:
n is an integer from 1 to about 4;
R₄ represents hydrogen, a C₁-C₆ alkyl group, a C₃-C₈ cycloalkyl group or a C₇-C₁₈ aralkyl group;
or a pharmaceutically acceptable salt, solvate, hydrate or N-oxide derivative thereof.

10. The method of claim 9, wherein the compound of Formula I is an N-oxide derivative.

11. The method of claim 9, wherein for the compound of Formula I Y represents —O— or 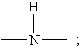
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 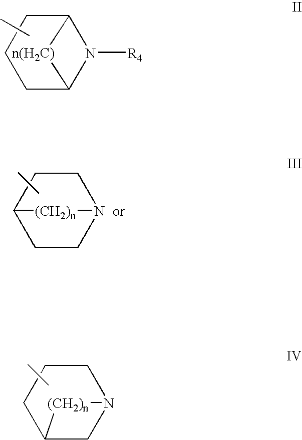
wherein:
n is 2 or 3; and
R₄ represents a C₁-C₆ alkyl group.

12. The method of claim 9, wherein for the compound of Formula I R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

13. The method of claim 1, wherein the compound having 5-HT₃ receptor agonist activity is represented by Formula V: 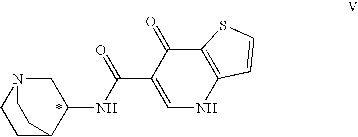
or a pharmaceutically acceptable salt, solvate or hydrate thereof.

14. The method of claim 13, wherein for the compound of Formula V the asterisked carbon atom is in the (R) configuration.

15. The method of claim 14, wherein the compound of Formula V is in the form of the monohydrochloride salt.

16. A method of treating a gastrointestinal motility disorder in a subject in need of treatment comprising coadministering to said subject:
a) a first amount of a compound having 5-HT₃ receptor agonist activity or a pharmaceutically acceptable salt, hydrate or solvate thereof; and
b) a second amount of at least one gastric acid suppressing agent, wherein the gastric acid suppressing agent is a proton pump inhibitor or a pharmaceutically acceptable salt, hydrate or solvate thereof,
wherein the first and second amounts together comprise a therapeutically effective amount.

17. The method of claim 16, wherein the proton pump inhibitor is selected from the group consisting of esomeprazole, omeprazole, lansoprazole, rabeprazole and pantoprazole.

18. The method of claim 16, wherein the compound having 5-HT₃ agonist activity is (R)—N-1-azabicyclo[2.2.2]oct-3-yl-4,7-dihydro-7-oxothieno[3,2-b]pyridine-6-carboxamide or a pharmaceutically acceptable salt, hydrate or solvate thereof.

19. The method of claim 18, wherein the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide.

20. A method of treating a gastrointestinal motility disorder in a subject in need of treatment comprising coadministering to said subject:
a) a first amount of a compound having 5-HT₃ receptor agonist activity or a pharmaceutically acceptable salt, hydrate or solvate thereof; and
b) a second amount of at least one gastric acid suppressing agent, wherein the gastric acid suppressing agent is an H₂ receptor antagonist or a pharmaceutically acceptable salt, hydrate or solvate thereof,
wherein the first and second amounts together comprise a therapeutically effective amount.

21. The method of claim 20, wherein the H₂ receptor antagonist is selected from the group consisting of nizatidine, ranitidine, famotidine, roxatidine and cimetidine.

22. The method of claim 20, wherein the compound having 5-HT₃ agonist activity is (R)—N-1-azabicyclo[2.2.2]oct-3-yl-4,7-dihydro-7-oxothieno[3,2-b]pyridine-6-carboxamide or a pharmaceutically acceptable salt, hydrate or solvate thereof.

23. The method of claim 22, wherein the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide.

24. A method of treating a gastrointestinal motility disorder in a subject in need of treatment comprising coadministering to said subject:
a) a first amount of a compound having 5-HT₃ receptor agonist activity or a pharmaceutically acceptable salt, hydrate or solvate thereof; and
b) a second amount of at least one gastric acid suppressing, wherein the gastric acid suppressing agent is an acid pump antagonist selected from the group consisting of: soraprazan, AZD0865, YH1885 and CS-526,
wherein the first and second amounts together comprise a therapeutically effective amount.

25. The method of claim 24, wherein the compound having 5-HT₃ agonist activity is (R)—N-1-azabicyclo[2.2.2]oct-3-yl-4,7-dihydro-7-oxothieno[3,2-b]pyridine-6-carboxamide or a pharmaceutically acceptable salt, hydrate or solvate thereof.

26. The method of claim 25, wherein the compound having 5-HT₃ agonist activity is the monohydrochloride salt of (R)—N-1-azabicyclo[2.2.2]oct-3-yl-4,7-dihydro-7-oxothieno[3,2-b]pyridine-6-carboxamide.

27. A method of treating a gastrointestinal motility disorder in a subject in need of treatment comprising coadministering to said subject:
a) a first amount of a compound having 5-HT₃ receptor agonist activity wherein the compound having 5-HT₃ receptor agonist activity is represented by Formula VI or a pharmaceutically acceptable salt, solvate or hydate thereof: 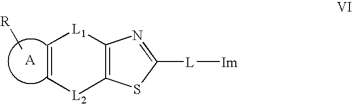
wherein:
R represents hydrogen, halogen, hydroxyl, a C₁-C₆ alkoxy group, carboxy, a C₁-C₆ alkoxycarbonyl group, nitro, amino, cyano or protected hydroxyl; 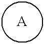
is a phenyl ring or a naphthalene ring;
L is a direct bond or a C₁-C₆ alkylene group;
L₁ and L₂ are defined so that one is a direct bond and the other is:
a) a C₁-C₆ alkylene group optionally containing and interrupting oxygen or sulfur atom therein;
b) an oxygen atom or sulfur atom; or
c) a C₁-C₆ alkenylene group;
Im represents a group having the formula: 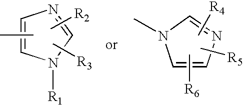
wherein:
R₁-R6 are the same or different each representing hydrogen or a C₁-C₆ alkyl group; and
b) a second amount of at least one gastric acid suppressing agent, wherein the first and second amounts together comprise a therapeutically effective amount.

28. The method of claim 27, wherein for the compound of Formula VI, 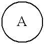
is a phenyl ring, L₁ is a direct bond and L₂ is an alkylene group or alkenylene group.

29. A method of claim 27, wherein the compound having 5-HT₃ receptor agonist activity is represented by Formula VII: 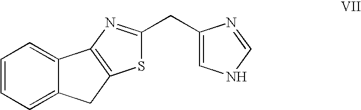
or a pharmaceutically acceptable salt, solvate or hydrate thereof.

30. The method of claim 27, wherein the gastric acid suppressing agent is a proton pump inhibitor, an H₂ receptor antagonist or a pharmaceutically acceptable salt, hydrate or solvate thereof.

31. The method of claim 30, wherein the gastric acid suppressing agent is a proton pump inhibitor.

32. The method of claim 31, wherein the proton pump inhibitor is selected from the group consisting of esomeprazole, omeprazole, lansoprazole, rabeprazole and pantoprazole.

33. The method of claim 30, wherein the gastric acid suppressing agent is an H₂ receptor antagonist.

34. The method of claim 33, wherein the H₂ receptor antagonists is selected from the group consisting of nizatidine, ranitidine, famotidine, roxatidine and cimetidine.

35. The method of claim 27, wherein the gastric acid suppressing agent is an acid pump antagonist.

36. The method of claim 35, wherein the acid pump antagonist is selected from the group consisting of soraprazan, AZD0865, YH1885 and CS-526.

37. The method of claim 26, wherein the gastrointestinal motililty disorder is gastroparesis.

38. The method of claim 27, wherein the subject is a human.

39. A method of treating GERD in a subject in need of treatment comprising coadministering to said subject:
a) a first amount of a compound represented by Formula I: 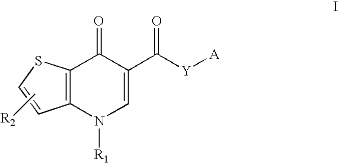
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 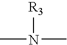
wherein R₃ represents hydrogen or a C₁-C₆ alkyl group; and
A is represented by 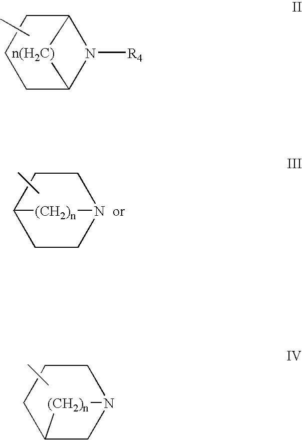
wherein:
n is an integer from 1 to about 4;
R₄ represents hydrogen, a C₁-C₆ alkyl group, a C₃-C₈ cycloalkyl group or a C₇-C₁₈ aralkyl group;
or a pharmaceutically acceptable salt, solvate, hydrate or N-oxide thereof; and
b) a second amount of at least one gastric acid suppressing agent or a
pharmaceutically acceptable salt, hydrate or solvate thereof, wherein the first and second amounts together comprise a therapeutically effective amount.

40. The method of claim 39, wherein the GERD is nocturnal GERD.

41. The method of claim 39, wherein the gastric acid suppressing agent is a proton pump inhibitor, an H₂ receptor antagonist or a pharmaceutically acceptable salt, hydrate or solvate thereof.

42. The method of claim 41, wherein the gastric acid suppressing agent is a proton pump inhibitor.

43. The method of claim 42, wherein the proton pump inhibitor is selected from the group consisting of esomeprazole, omeprazole, lansoprazole, rabeprazole and pantoprazole.

44. The method of claim 41, wherein the gastric acid suppressing agent is an H₂ receptor antagonist.

45. The method of claim 44, wherein the H₂ receptor antagonist is selected from the group consisting of nizatidine, ranitidine, famotidine, roxatidine and cimetidine.

46. The method of claim 39, wherein the gastric acid suppressing agent is an acid pump antagonist.

47. The method of claim 46, wherein the acid pump antagonist is selected from the group consisting of soraprazan, AZD0865, YH1885 and CS-526.

48. The method of claim 39 wherein the compound of Formula I is an N-oxide derivative.

49. The method of claim 39, wherein for the compound of Formula I Y represents —O=13 or 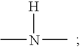
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 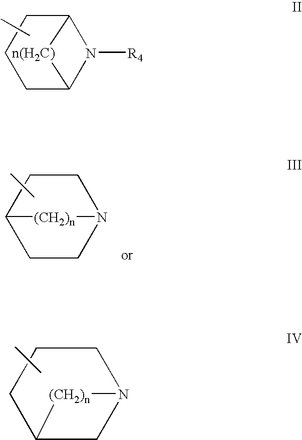
wherein:
n is 2 or 3; and
R₄ represents a C₁-C₆ alkyl group.

50. The method of claim 39, wherein for the compound of Formula I, R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

51. A method of treating GERD in a subject in need of treatment comprising coadministering to said subject:
a) a first amount of a compound represented by Formula V: 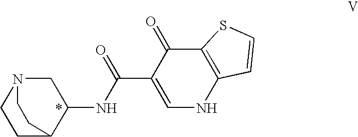
or a pharmaceutically acceptable salt, solvate or hydrate thereof, and
b) a second amount of at least one gastric acid suppressing agent or a
pharmaceutically acceptable salt, hydrate or solvate thereof, wherein the first and second amounts together comprise a therapeutically effective amount.

52. The method of claim 5 1, wherein the GERD is nocturnal GERD.

53. The method of claim 5 1, wherein for the compound of Formula V the asterisked carbon atom is in the (R) configuration.

54. The method of claim 53, wherein the compound of Formula V is in the form of S the monohydrochloride salt.

55. The method of claim 5 1, wherein the gastric acid suppressing agent is a proton pump inhibitor, an H₂ receptor antagonist or a pharmaceutically acceptable salt, hydrate or solvate thereof.

56. The method of claim 55, wherein the gastric acid suppressing agent is a proton pump inhibitor selected from the group consisting of esomeprazole, omeprazole, lansoprazole, rabeprazole and pantoprazole.

57. The method of claim 55, wherein the gastric acid suppressing agent is an H₂ receptor antagonist selected from the group consisting of nizatidine, ranitidine, famotidine, roxatidine and cimetidine.

58. The method of claim 51, wherein the gastric acid suppressing agent is an acid pump antagonist or a pharmaceutically acceptable salt, hydrate or solvate thereof.

59. The method of claim 58, wherein the acid pump antagonist is selected from the group consisting of soraprazan, AZD0865, YH1885 and CS-526.

60. A method of treating GERD in a subject in need of treatment comprising coadministering to said subject:
a) a first amount of a compound represented by Formula VI or a pharmaceutically acceptable salt, solvate or hydate thereof: 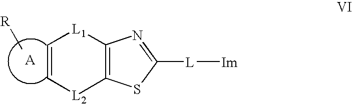
wherein:
R represents hydrogen, halogen, hydroxyl, a C₁-C₆ alkoxy group, carboxy, a C₁-C₆ alkoxycarbonyl group, nitro, amino, cyano or protected hydroxyl; 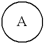
is a phenyl ring or a naphthalene ring;
L is a direct bond or a C₁-C₆ alkylene group;
L₁ and L₂ are defined so that one is a direct bond and the other is:
a) a C₁-C₆ alkylene group optionally containing and interrupting oxygen or sulfur atom therein;
b) an oxygen atom or sulfur atom; or
c) a C₁-C₆ alkenylene group;
Im represents a group having the formula: 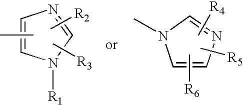
wherein:
R₁-R₆ are the same or different each representing hydrogen or a C₁-C₆ alkyl group; and
b) a second amount of at least one gastric acid suppressing agent,
wherein the first and second amounts together comprise a therapeutically effective amount.

61. The method of claim 60, wherein the GERD is nocturnal GERD.

62. The method of claim 60, wherein the gastric acid suppressing agent is a proton pump inhibitor, an H₂ receptor antagonist or a pharmaceutically acceptable salt, hydrate or solvate thereof.

63. The method of claim 62, wherein the gastric acid suppressing agent is a proton pump inhibitor selected from the group consisting of esomeprazole, omeprazole, lansoprazole, rabeprazole and pantoprazole.

64. The method of claim 62, wherein the gastric acid suppressing agent is an H₂ receptor antagonist selected from the group consisting of nizatidine, ranitidine, famotidine, roxatidine and cimetidine.

65. The method of claim 60, wherein the gastric acid suppressing agent is an acid pump antagonist or a pharmaceutically acceptable salt, hydrate or solvate thereof.

66. The method of claim 65, wherein the acid pump antagonist is selected from the group consisting of soraprazan, AZD0865, YH1885 and CS-526.

67. The method of claim 60, wherein for the compound of Formula VI, 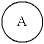
is a phenyl ring, L₁ is a direct bond and L₂ is an alkylene group or alkenylene group.

68. The method of claim 60, wherein the compound Formula VI is represented by Formula VII: 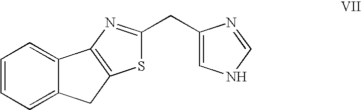
or a pharmaceutically acceptable salt, solvate or hydrate thereof.

69. The method of claim 68, wherein the gastric acid suppressing agent is a proton pump inhibitor selected from the group consisting of esomeprazole, omeprazole, lansoprazole, rabeprazole and pantoprazole.

70. The method of claim 68, wherein the gastric acid suppressing agent is an H₂ receptor antagonist selected from the group consisting of nizatidine, ranitidine, famotidine, roxatidine and cimetidine.

71. A pharmaceutical composition comprising:
a) a first amount of a compound having 5-HT₃ receptor agonist activity or a pharmaceutically acceptable salt, hydrate or solvate thereof; and
b) a second amount of at least one gastric acid suppressing agent,
wherein the first and second amounts together comprise a therapeutically effective amount.

72. The pharmaceutical composition of claim 71, wherein the gastric acid suppressing agent is a proton pump inhibitor, an H₂ receptor antagonist or a pharmaceutically acceptable salt, hydrate or solvate thereof.

73. The pharmaceutical composition of claim 72, wherein the gastric acid suppressing agent is a proton pump inhibitor selected from the group consisting of esomeprazole, omeprazole, lansoprazole, rabeprazole and pantoprazole.

74. The pharmaceutical composition of claim 72, wherein the gastric acid suppressing agent is an H₂ receptor antagonist selected from the group consisting of nizatidine, ranitidine, famotidine, roxatidine and cimetidine.

75. The pharmaceutical composition of claim 71, wherein the gastric acid suppressing agent is an acid pump antagonist or a pharmaceutically acceptable salt, hydrate or solvate thereof.

76. The pharmaceutical composition of claim 75, wherein the acid pump antagonist is selected from the group consisting of soraprazan, AZD0865, YH1 885 and CS-526.

77. The pharmaceutical composition of claim 71, wherein the compound having 5-HT₃ receptor agonist activity is represented by Formula I: 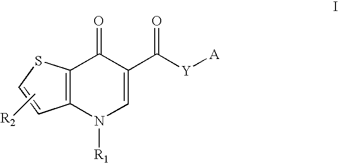
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 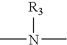
wherein R₃ represents hydrogen or a C₁-C₆ alkyl group; and
A is represented by 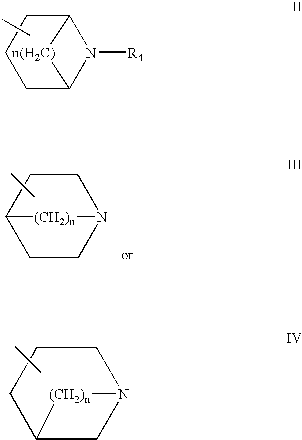
wherein:
n is an integer from 1 to about 4;
R₄ represents hydrogen, a C₁-C₆ alkyl group, a C₃-C₈ cycloalkyl group or a C₇-C₁₈ aralkyl group;
or a pharmaceutically acceptable salt, solvate, hydrate or N-oxide derivative thereof.

78. The pharmaceutical composition of claim 77, wherein the compound of Formula I is an N-oxide derivative.

79. The pharmaceutical composition of claim 77, wherein for the compound of Formula I
Y represents —O— or 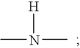
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 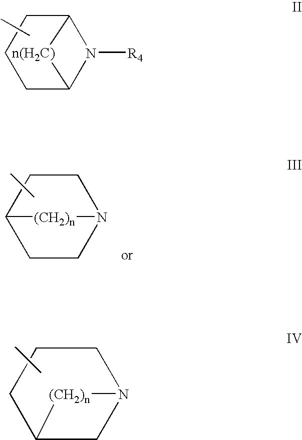
wherein:
n is 2 or 3; and
R₄represents a C₁-C₆ alkyl group.

80. The pharmaceutical composition of claim 77, wherein for the compound of Formula I, R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

81. The pharmaceutical composition of claim 71, wherein the compound having 5-HT₃ receptor agonist activity is represented by Formula V: 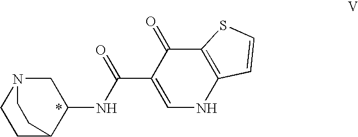
or a pharmaceutically acceptable salt, solvate or hydrate thereof.

82. The pharmaceutical composition of claim 81, wherein for the compound of Formula V the asterisked carbon atom is in the (R) configuration.

83. The pharmaceutical composition of claim 82, wherein the compound of Formula V is in the form of the monohydrochloride salt.

84. A kit comprising a compound having 5-HT₃ receptor agonist activity, instructions for use with at least one gastric acid suppressing agent and optionally a device for administering the compounds.

85. The kit of claim 84, wherein the gastric acid suppressing agent is a proton pump inhibitor, an H₂ receptor antagonist or a pharmaceutically acceptable salt, hydrate or solvate thereof.

86. The kit of claim 85, wherein the gastric acid suppressing agent is a proton pump inhibitor.

87. The kit of claim 86, wherein the proton pump inhibitor is selected from the group consisting of esomeprazole, omeprazole, lansoprazole, rabeprazole and pantoprazole.

88. The kit of claim 85, wherein the gastric acid suppressing agent is an H₂ receptor antagonist.

89. The kit of claim 88, wherein the H₂ receptor antagonist is selected from the group consisting of nizatidine, ranitidine, famotidine, roxatidine and cimetidine.

90. The kit of claim 84, wherein the 5-HT₃ receptor agonist is present in the kit in a sub-therapeutic dose.

91. The kit of claim 84, wherein the instructions specify that the gastric acid suppressing agent is used in a sub-therapeutic dose.

92. The kit of claim 84, wherein the compound having 5-HT₃ receptor agonist activity is represented by Formula I: 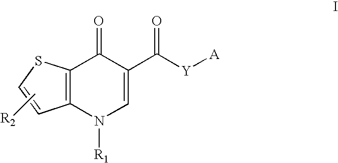
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 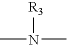
wherein R₃ represents hydrogen or a C₁-C₆ alkyl group; and
A is represented by 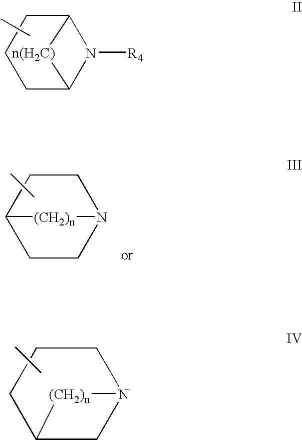
wherein:
n is an integer from 1 to about 4;
R₄ represents hydrogen, a C₁-C₆ alkyl group, a C₃-C₈ cycloalkyl group or a C₇-C₁₈ aralkyl group;
or a pharmaceutically acceptable salt, solvate, hydrate or N-oxide derivative thereof.

93. The kit of claim 92, wherein the compound of Formula I is an N-oxide derivative.

94. The kit of claim 92, wherein for the compound of Formula I Y represents —O— or 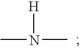
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 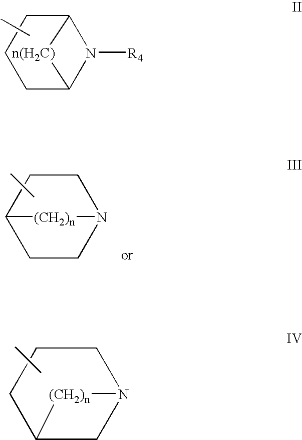
wherein:
n is 2 or 3; and
R₄ represents a C₁-C₆ alkyl group.

95. The kit of claim 92, wherein for the compound of Formula I R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

96. The kit of claim 92, wherein the compound having 5-HT₃ receptor agonist activity is represented by Formula V: 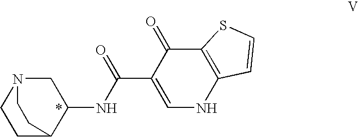
or a pharmaceutically acceptable salt, solvate or hydrate thereof.

97. The kit of claim 96, wherein for the compound of Formula V the asterisked carbon atom is in the (R) configuration.

98. The kit of claim 97, wherein the compound of Formula V is in the form of the monohydrochloride salt.

99. A kit comprising at least one compound which is a gastric acid suppressing agent, instructions for use with a compound having 5-HT₃ receptor agonist activity, and optionally a device for administering the compounds.

100. The kit of claim 99, wherein the gastric acid suppressing agent is a proton pump inhibitor, an H₂ receptor antagonist or a pharmaceutically acceptable salt, hydrate or solvate thereof.

101. The kit of claim 100, wherein the gastric acid suppressing agent is a proton pump inhibitor.

102. The kit of claim 101, wherein the proton pump inhibitor is selected from the group consisting of esomeprazole, omeprazole, lansoprazole, rabeprazole and pantoprazole.

103. The kit of claim 100, wherein the gastric acid suppressing agent is an H₂ receptor antagonist.

104. The kit of claim 103, wherein the H₂ receptor antagonist is selected from the group consisting of nizatidine, ranitidine, famotidine, roxatidine and cimetidine.

105. The kit of claim 100, wherein the instructions specify that the compound having 5-HT₃ receptor agonist activity is used in a sub-therapeutic dose.

106. The kit of claim 100, wherein the gastric acid suppressing agent is present in the kit in a sub-therapeutic dose.

107. The kit of claim 99, wherein the compound having 5-HT₃ receptor agonist activity is represented by Formula I: 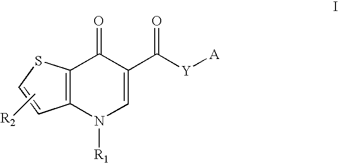
wherein:
R₁ represents hydrogen, a C₁-C₆ alkyl group, a C₂-C₆ alkenyl group, a C₂-C₆ alkynyl group, a C₃-C₈ cycloalkyl group, a C₆-C ₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 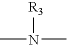
wherein R₃ represents hydrogen or a C₁-C₆ alkyl group; and
A is represented by 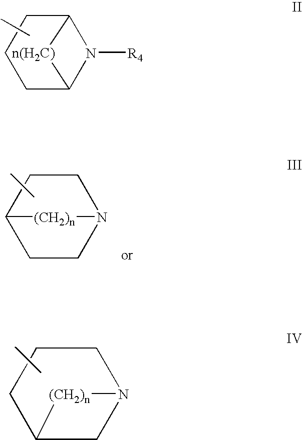
wherein:
n is an integer from 1 to about 4;
R₄ represents hydrogen, a C₁-C₆alkyl group, a C₃-C₈ cycloalkyl group or a C₇-C₁₈ aralkyl group;
or a pharmaceutically acceptable salt, solvate, hydrate or N-oxide derivative thereof.

108. The kit of claim 107, wherein the compound of Formula I is an N-oxide derivative.

109. The kit of claim 107, wherein for the compound of Formula I
Y represents —O— or 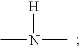
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 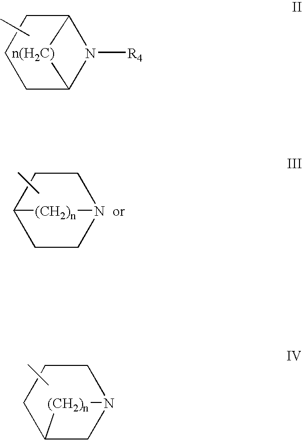
wherein:
n is 2 or 3; and
R₄ represents a C₁-C₆ alkyl group.

110. The kit of claim 107, wherein for the compound of Formula I, R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

111. The kit of claim 107, wherein the compound having 5-HT₃ receptor agonist activity is represented by Formula V: 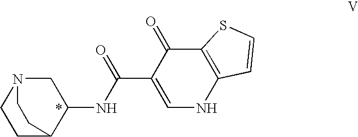
or a pharmaceutically acceptable salt, solvate or hydrate thereof.

112. The kit of claim 111, wherein for the compound of Formula V the asterisked carbon atom is in the (R) configuration.

113. The kit of claim 112, wherein the compound of Formula V is in the form of the monohydrochloride salt.

114. A kit comprising a compound having 5-HT₃ receptor agonist activity, at least one gastric acid suppressing agent, instructions for coadministering the compound having 5-HT₃ receptor agonist activity and the gastric acid suppressing agent, and optionally a device for administering the compounds.

115. The kit of claim 114, wherein the gastric acid suppressing agent is a proton pump inhibitor, an H₂ receptor antagonist or a pharmaceutically acceptable salt, hydrate or solvate thereof.

116. The kit of claim 115, wherein the gastric acid suppressing agent is a proton pump inhibitor.

117. The kit of claim 116, wherein the proton pump inhibitor is selected from the group consisting of esomeprazole, omeprazole, lansoprazole, rabeprazole and pantoprazole.

118. The kit of claim 115, wherein the gastric acid suppressing agent is an H₂ receptor antagonist.

119. The kit of claim 118, wherein the H₂ receptor antagonist is selected from the group consisting of nizatidine, ranitidine, famotidine, roxatidine and cimetidine.

120. The kit of claim 114, wherein the compound having 5-HT₃ receptor agonist activity is present in the kit in a sub-therapeutic dose.

121. The kit of claim 114, wherein the gastric acid suppressing agent is present in the kit in a sub-therapeutic dose.

122. The kit of claim 114, wherein the compound having 5-HT₃ receptor agonist activity is represented by Formula I: 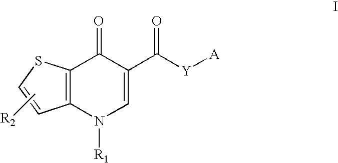
wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or 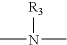
wherein R₃ represents hydrogen or a C₁-C₆ alkyl group; and
A is represented by 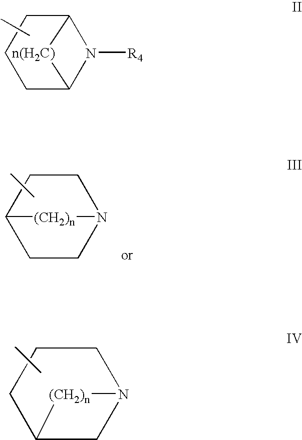
wherein:
n is an integer from 1 to about 4;
R₄ represents hydrogen, a C₁-C₆ alkyl group, a C₃-C₈ cycloalkyl group or a C₇-C₁₈ aralkyl group;
or a pharmaceutically acceptable salt, solvate, hydrate or N-oxide derivative thereof.

123. The kit of claim 122, wherein the compound of Formula I is an N-oxide derivative.

124. The kit of claim 122, wherein for the compound of Formula I
Y represents —O— or 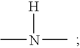
R₁ represents hydrogen, a C₁-C₆ alkyl group, a C₆-C₁₂ aryl group or a C₇-C₁₈ aralkyl group;
R₂ represents hydrogen, a C₁-C₆ alkyl group or halogen; and
A is represented by 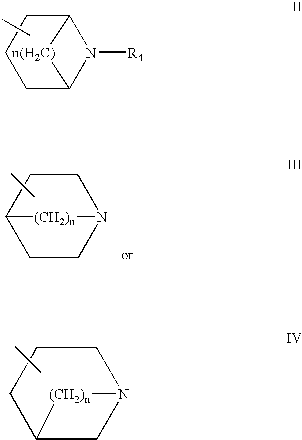
wherein:
n is 2 or 3; and
R₄represents a C₁-C₆ alkyl group.

125. The method of claim 122, wherein for the compound of Formula I R₁ represents hydrogen or a C₁-C₃ alkyl group, R₂ represents hydrogen, a C₁-C₃ alkyl group or halogen, R³ represents hydrogen, R⁴ represents a C₁-C₃ alkyl group and n is an integer of 2 or 3.

126. The kit of claim 122, wherein the compound having 5-HT₃ receptor agonist activity is represented by Formula V: 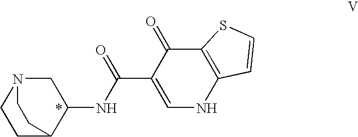
or a pharmaceutically acceptable salt, solvate or hydrate thereof.

127. The kit of claim 125, wherein for the compound of Formula V the asterisked carbon atom is in the (R) configuration.

128. The kit of claim 127, wherein the compound of Formula V is in the form of the monohydrochloride salt.

129. A method of treating nocturnal GERD in a subject in need thereof comprising administering a therapeutically effective amount of a compound having 5-HT₃ receptor agonist activity or a pharmaceutically acceptable salt, hydrate or solvate thereof.

130. The method of claim 129, wherein the compound having 5-HT₃ receptor agonist activity is represented by Formula V: 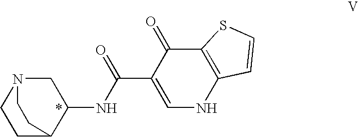
or a pharmaceutically acceptable salt, solvate or hydrate thereof.

131. A method of increasing esophageal motility in a subject in need thereof comprising administering a therapeutically effective amount of a compound having 5-HT₃ receptor agonist activity or a pharmaceutically acceptable salt, hydrate or solvate thereof.

132. The method of claim 131, wherein the compound having 5-HT₃ receptor agonist activity is represented by Formula V: 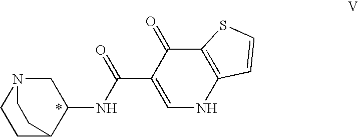
or a pharmaceutically acceptable salt, solvate or hydrate thereof.

133. A method of increasing esophageal motility in a subject in need of treatment comprising coadministering to said subject:
a) a first amount of a compound having 5-HT₃ receptor agonist activity or a pharmaceutically acceptable salt, hydrate or solvate thereof; and
b) a second amount of at least one gastric acid suppressing agent or a pharmaceutically acceptable salt, hydrate or solvate thereof,
wherein the first and second amounts together comprise a therapeutically effective amount.

134. The method of claim 133, wherein the compound having 5-HT₃ receptor agonist activity is represented by Formula V: 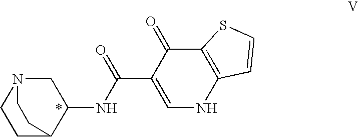
or a pharmaceutically acceptable salt, solvate or hydrate thereof.
